(19) United States
(12) Patent Application Publication
(10) Pub. No.: US 2024/0132573 A1
(43) Pub. Date: Apr. 25, 2024

(54) MODIFIED SERPINS FOR THE TREATMENT OF BRADYKININ-MEDIATED DISEASE

(71) Applicant: PRECLINICS GESELLSCHAFT FÜR PRÄKLINISCHE FORSCHUNG MBH, Potsdam (DE)

(72) Inventors: Coen MAAS, Utrecht (NL); Steven DE MAAT, Utrecht (NL)

(21) Appl. No.: 18/530,002

(22) Filed: Dec. 5, 2023

Related U.S. Application Data
(62) Division of application No. 16/487,858, filed on Aug. 22, 2019, now Pat. No. 11,851,474.

(30) Foreign Application Priority Data
Feb. 23, 2017 (EP) 17157527.7

(51) Int. Cl. C07K 14/81

(52) U.S. Cl. CPC C07K 14/8125; A61K 38/00

(57) ABSTRACT

A method of treating a bradykinin-mediated disease, wherein the method includes administering to a subject in need thereof an effective amount of a modified α1 antitrypsin including a reactive center loop (RCL), wherein the residues P4-P1 of the RCL are selected from the group consisting of SMTR (SEQ ID NO: 12), SEAR (SEQ ID NO: 18), SMDR (SEQ ID NO: 23), SLGR (SEQ ID NO: 24), SKGR (SEQ ID NO: 25) and SMHR (SEQ ID NO: 27), wherein the modified al antitrypsin demonstrates increased inhibition of plasma kallikrein (PK) as compared to the corresponding unmodified α1 antitrypsin, and wherein the modified α1-antitrypsin more strongly inhibits PK than the modified α1 antitrypsin inhibits either one of thrombin or APC.

### FIELD OF THE INVENTION

The present invention relates to the field of medicine and pharmacy, in particular to the field of biopharmaceuticals for use in the treatment of bradykinin-mediated disease. More specifically, the invention relates to serine protease inhibitors (serpin) molecules modified to have increased specificity for bradykinin-generating proteases of the plasma contact system, particularly plasma kallikrein (PK) and active forms of factor XII (FXII) and/or plasmin.

### REFERENCE TO SEQUENCE LISTING

The present application is being filed along with a Sequence Listing in electronic format. The Sequence Listing is provided as a file entitled 58697113_1.XML, created and last modified on Dec. 5, 2023, which is 432 Kb in size. The information in the electronic format of the Sequence Listing is incorporated herein by reference in its entirety.

### BACKGROUND ART

α1-antitrypsin (α1AT) is a naturally occurring serine protease inhibitor (serpin) in human blood. Its natural function is to inhibit amongst others the inflammatory enzyme leucocyte elastase. To execute this function, it acts as a molecular mouse-trap: the serpin contains a loop with a specific amino acid sequence that is recognized and cleaved by elastase. This generates a stable (SDS and reduction resistant) complex between α1AT and elastase that renders the latter inactive. This inactivation mechanism is a common feature of serpins and the specific amino acid sequence of a serpin's reactive center loop (RCL) determines which serine protease enzyme recognizes the specific serpin and is inhibited by it.

In hereditary angioedema (HAE), overproduction of the inflammatory peptide bradykinin causes painful and dangerous attacks of tissue swelling. Bradykinin is produced by enzymes of the plasma contact system (FXIIa, PK). These enzymes are controlled by the serpin C1-esterase inhibitor (C1INH). Hence, in HAE, congenital deficiency in C1INH leads to uncontrolled bradykinin production. In a similar manner, neutralizing antibodies against C1INH cause acquired angioedema.

We recently identified plasmin as a new player in the bradykinin forming cascade (de Maat et al. J Allergy Clin Immunol. 2016 Apr. 6. pii: 80091-6749(16)30006-9). In addition, the pathological consequences of excessive bradykinin production extend beyond angioedema: in severe allergic reactions, bradykinin is a critical disease mediator (Oschatz et al. Immunity. 2011 Feb. 25; 34(2):258-68; Sala-Cunill et al. J Allergy Clin Immunol. 2015 April; 135(4):1031-43).

It has been previously described that the α1AT Pittsburgh variant has a single point mutation (M358R, numbering based upon mature protein sequence of natural occurring α1AT) that significantly alters its specificity (Scott et al. J Clin Invest. 1986 February; 77(2):631-4). From an inhibitor of elastase and trypsin, it has now become a strong inhibitor of the contact system enzymes but also of activated protein C (APC) (Heeb et al 1990 J Biol Chem February 5; 265(4):2365-9) and thrombin, the latter causing bleeding in patients carrying this variant.

WO2006/090282 discloses variants of α1AT that are modified for increased substrate specificity towards the human tissue kallikrein 14 (hK14) protease, a kallikrein the expression of which is correlated with poor prognosis for breast and prostate cancers.

For HAE, protein re-supplementation therapy (plasma purified or recombinant C1INH) is the golden standard (Cicardi et al. J. Invest. Allergol Clin Immunol 2016 January; 1; 26 (4):212-22). This therapy is not ideal because of the relatively short circulating half-life of C1INH (Caliezi et al Pharmacol Rev 2000 March; 52(1):91-112). For example, plasma derived C1INH has a half-life time of 32.7 hours (Bernstein et al., Ann Allergy Asthma Immunol. 2010 August; 105(2):149-54) and the only recombinant C1INH currently available, Ruconest®, has a half-life of only three hours (van Doorn et al J Allergy Clin Immunol 2005 October; 116(4):876-83) whereas for example α1AT has a half-life of 4-5 days (Archibald et al Proc. Natl, Acad Sci 1990 87; 5178-5182). Furthermore, on a molecular basis, C1INH is a kinetically unfavorable serpin. It has weak inhibitory properties towards contact system enzymes (Scott et al J Clin Invest 1986 February; 77(2):631-4).

This becomes clear in the clinic: the large majority patients (>95%) that suffer from angioedema are without C1INH deficiency (idiopathic angioedema). There is no therapy for these patients, but bradykinin is strongly implicated as disease mediator (Cugno et al 2017 Clin Exp Allergy. 2017 January; 47(1):139-140). In these patients, C1INH is not able to maintain in control of the contact system.

It is an object of the present invention to provide for novel modified serpins that are useful in the treatment of bradykinin-mediated diseases and that address the shortcomings of C1INH.

### SUMMARY OF THE INVENTION

In a first aspect the invention relates to a modified serpin that has mutations in one or more of the P4, P3, P2, P1, and P1′ residues of its reactive center loop (RCL), which mutations increase the inhibition of plasma kallikrein (PK) as compared to the corresponding unmodified serine protease inhibitors (serpin). Preferably the modified serpin more strongly inhibits PK than the serpin inhibits thrombin, and also preferably, the modified serpin more strongly inhibits PK than the serpin inhibits activated protein C (APC). The modified serpin preferably is for use in the treatment of a bradykinin-mediated disease.

Preferably in a modified serpin of the invention, the mutations increase the inhibition of at least one of an active form of Factor XII and plasmin as compared to the corresponding unmodified serpin, and wherein, preferably, the serpin inhibits at least one of an active form of FXII and PK as strong or stronger than they are inhibited by C1 esterase inhibitor. A modified serpin of the invention is preferably characterized in that the minimal amount of the serpin that inhibits PK activity by at least 50%, is an amount that inhibits thrombin activity by no more than 15%. More preferably, the minimal amount of the serpin that inhibits PK activity by at least 50%, is an amount that inhibits at least one of: a) plasmin by at least 15%; b) an active form of FXII by at least 15%; and, c) APC by no more than 15%.

A modified serpin of the invention preferably is a serpin comprising an RCL wherein the P1 residue is lysine (K) or arginine (R), the P2 residue is not proline (P) and wherein the P4 residue is serine (S). More preferably, the serpin comprises an RCL wherein the residues P4-P1 of the RCL have an amino acid sequence selected from the group consisting of SMTR (SEQ ID NO: 12), SGQR (SEQ ID NO: 13), SVTR (SEQ ID NO: 14), SATR (SEQ ID NO: 15), SFNR (SEQ ID NO: 16), SWKK (SEQ ID NO: 17), SEAR (SEQ ID NO: 18), SVVK (SEQ ID NO: 19), SDYK (SEQ ID NO: 20), SVRK (SEQ ID NO: 21), SPRR (SEQ ID NO: 22), SMDR (SEQ ID NO: 23), SLGR (SEQ ID NO: 24), SKGR (SEQ ID NO: 25), SGNR (SEQ ID NO: 26), SMHR (SEQ ID NO: 27) and SLLR (SEQ ID NO: 28). A modified serpin of the invention further preferably has a mutation whereby the P1′ residue of its reactive center loop (RCL) is selected from the group consisting of: Q, F, H, R, K, C, L, Y, N, I, D, W, E, V, M, A, T and P, of which K, L, Y, I, D, E, A, T, M, R and V are preferred, and I and V are most preferred. A particularly preferred modified serpin of the invention is a serpin wherein the residues P4-P1 of the RCL have the amino acid sequence SLLR (SEQ ID NO: 28) and wherein the P1′ residue is selected from the group consisting of: Q, F, H, R, K, C, L, Y, N, I, D, W, E, V, M, A, T and P, of which K, L, Y, I, D, E, A, T, M, R and V are preferred, and I and V are most preferred.

A modified serpin according to the invention preferably is a serpin comprising an amino acid sequence that has at least 70% sequence identity to the sequence of a wild-type serpin and wherein preferably, the wild type serpin is selected from the group consisting of α1-antitrypsin (α1AT) (SERPINA1), α1-antichymotrypsin (SERPINA3), C1-esterase inhibitor (SERPING1), a2-antiplasmin (SERPINF2), antithrombin (ATIII) (SERPINC1), heparin cofactor II (HCII) (SERPIND1), protein C inhibitor (PCI) (SERPINA5), Kallistatin (SERPINA4), Plasminogen activator inhibitor (SERPINE1), Protease nexin 1 (SERPINE2) and Protein Z-dependent protease inhibitor (SERPINA10), of which α1AT is most preferred. A modified serpin of the invention further preferably is a serpin that has been modified to reduce the susceptibility to oxidation.

In a second aspect, the invention relates to a modified serpin of the invention for use in the treatment of a bradykinin-mediated disease. Preferably, the serpin is for use in the treatment of a bradykinin-mediated disease selected from the group consisting of hereditary angioedema, idiopathic angioedema, rheumatoid arthritis, Crohn's disease, ulcerative colitis, lupus, Alzheimer's disease, septic shock, burn injury, brain ischemia/reperfusion injury, cerebral edema, diabetic retinopathy, diabetic nephropathy, macular edema, vasculitis, arterial or venous thrombosis, thrombosis associated with ventricular assist devices or stents, heparin-induced thrombocytopenia with thrombosis, thromboembolic disease, and coronary heart disease with unstable angina pectoris, edema, eye disease, gout, intestinal bowel disease, oral mucositis, neuropathic pain, inflammatory pain, spinal stenosis-degenerative spine disease, post-operative ileus, aortic aneurysm, osteoarthritis, hereditary angioedema, pulmonary embolism, stroke, head trauma or peri-tumor brain edema, sepsis, acute middle cerebral artery (MCA) ischemic event (stroke), restenosis (e.g., after angioplasty), systemic lupus erythematosis nephritis, an autoimmune disease, an inflammatory disease for example (chronic) auto-inflammatory urticaria, a cardiovascular disease, a neurological disease, a disease associated with protein misfolding, a disease associated with angiogenesis, hypertensive nephropathy and diabetic nephropathy, allergic and respiratory diseases (e.g. anaphylaxis, asthma, chronic obstructive pulmonary disease, acute respiratory distress syndrome, cystic fibrosis, persistent, rhinitis) and tissue injuries (e.g. burn or chemical injury).

In a third aspect, the invention relates to a gene therapy vector comprising a nucleotide sequence encoding a modified serpin according to the invention. Preferably the gene therapy vector is for use in the treatment of a bradykinin-mediated disease, wherein, preferably, the bradykinin-mediated disease is selected from the group consisting of hereditary angioedema, idiopathic angioedema, rheumatoid arthritis, Crohn's disease, ulcerative colitis, lupus, Alzheimer's disease, septic shock, burn injury, brain ischemia/reperfusion injury, cerebral edema, diabetic retinopathy, diabetic nephropathy, macular edema, vasculitis, arterial or venous thrombosis, thrombosis associated with ventricular assist devices or stents, heparin-induced thrombocytopenia with thrombosis, thromboembolic disease, and coronary heart disease with unstable angina pectoris, edema, eye disease, gout, intestinal bowel disease, oral mucositis, neuropathic pain, inflammatory pain, spinal stenosis-degenerative spine disease, post-operative ileus, aortic aneurysm, osteoarthritis, hereditary angioedema, pulmonary embolism, stroke, head trauma or peri-tumor brain edema, sepsis, acute middle cerebral artery (MCA) ischemic event (stroke), restenosis (e.g., after angioplasty), systemic lupus erythematosis nephritis, an autoimmune disease, an inflammatory disease for example (chronic) auto-inflammatory urticaria, a cardiovascular disease, a neurological disease, a disease associated with protein misfolding, a disease associated with angiogenesis, hypertensive nephropathy and diabetic nephropathy, allergic and respiratory diseases (e.g. anaphylaxis, asthma, chronic obstructive pulmonary disease, acute respiratory distress syndrome, cystic fibrosis, persistent, rhinitis) and tissue injuries (e.g. burn or chemical injury).

In a fourth aspect, the invention pertains to a nucleic acid molecule comprising a nucleotide sequence encoding a modified serpin according to invention.

In a fifth aspect, the invention relates to a vector comprising a nucleotide sequence encoding a modified serpin according to invention. Preferably, the vector is a gene therapy vector.

In a sixth aspect, the invention pertains to a host cell comprising the vector of the invention, which host cell preferably expresses a modified serpin of the invention encoded by a nucleotide sequence in the vector.

In a seventh aspect, the invention relates to a pharmaceutical composition. The pharmaceutical composition comprises a pharmaceutically acceptable excipient and a modified serpin of the invention, a nucleic acid molecule of the invention, gene therapy vector of the invention or a host cell of the invention comprising the nucleic acid molecule.

### DESCRIPTION OF THE INVENTION

### Definitions

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithms (e.g., Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall lengths, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information on the World-Wide Web at ncbi.nlm.hih.gov.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

Alternative Conservative Amino Acid Residue Substitution Classes.

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative Physical and Functional Classifications of Amino Acid Residues.

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, |
| | W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, R, S, P |
| | and T |
| Flexible residues | Q, T, K, S, G, P, D, E, and R |

Nucleotide sequences encoding modified serpins of the invention may also be defined by their capability to hybridize with the nucleotide sequences of encoding modified serpins as exemplified herein, under moderate, or preferably under stringent hybridization conditions. Stringent hybridization conditions are herein defined as conditions that allow a nucleic acid sequence of at least about 25, preferably about 50 nucleotides, 75 or 100 and most preferably of about 200 or more nucleotides, to hybridise at a temperature of about 6500 in a solution comprising about 1 M salt, preferably 6×SSC or any other solution having a comparable ionic strength, and washing at 6500 in a solution comprising about 0.1 M salt, or less, preferably 0.2×SSC or any other solution having a comparable ionic strength. Preferably, the hybridization is performed overnight, i.e., at least for 10 hours and preferably washing is performed for at least one hour with at least two changes of the washing solution. These conditions will usually allow the specific hybridization of sequences having about 90% or more sequence identity.

Moderate conditions are herein defined as conditions that allow a nucleic acid sequences of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. Preferably, the hybridization is performed overnight, i.e., at least for 10 hours, and preferably washing is performed for at least one hour with at least two changes of the washing solution. These conditions will usually allow the specific hybridization of sequences having up to 50% sequence identity. The person skilled in the art will be able to modify these hybridization conditions in order to specifically identify sequences varying in identity between 50% and 90%.

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. The terms “expression vector” or expression construct” refer to nucleotide sequences that are capable of affecting expression of a gene in host cells or host organisms compatible with such sequences. These expression vectors typically include at least suitable transcription regulatory sequences and optionally, 3′ transcription termination signals. Additional factors necessary or helpful in effecting expression may also be present, such as expression enhancer elements. The expression vector will be introduced into a suitable host cell and be able to effect expression of the coding sequence in an in vitro cell culture of the host cell. The expression vector will be suitable for replication in the host cell or organism of the invention.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g., by the application of a chemical inducer.

The term “selectable marker” is a term familiar to one of ordinary skill in the art and is used herein to describe any genetic entity which, when expressed, can be used to select for a cell or cells containing the selectable marker. The term “reporter” may be used interchangeably with marker, although it is mainly used to refer to visible markers, such as green fluorescent protein (GFP). Selectable markers may be dominant or recessive or bidirectional.

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g., an mRNA) in a cell, operably linked to suitable regulatory regions (e.g., a promoter). A gene will usually comprise several operably linked fragments, such as a promoter, a 5′ leader sequence, a coding region and a 3′ non-translated sequence (3′ end) comprising a polyadenylation site. “Expression of a gene” refers to the process wherein a DNA region which is operably linked to appropriate regulatory regions, particularly a promoter, is transcribed into an RNA, which is biologically active, i.e., which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later.

The term “heterologous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous nucleic acids or proteins are not endogenous to the cell into which it is introduced, but has been obtained from another cell or synthetically or recombinantly produced. Generally, though not necessarily, such nucleic acids encode proteins that are not normally produced by the cell in which the DNA is transcribed or expressed. Similarly exogenous RNA encodes for proteins not normally expressed in the cell in which the exogenous RNA is present. Heterologous nucleic acids and proteins may also be referred to as foreign nucleic acids or proteins. Any nucleic acid or protein that one of skill in the art would recognize as heterologous or foreign to the cell in which it is expressed is herein encompassed by the term heterologous nucleic acid or protein. The term heterologous also applies to non-natural combinations of nucleic acid or amino acid sequences, i.e., combinations where at least two of the combined sequences are foreign with respect to each other.

The phrases “pharmaceutical or pharmacologically acceptable” refers to molecular entities and compositions that do not produce or produce acceptable adverse, allergic or other untoward reaction when administered to an animal, such as, for example, a human, as appropriate. Whether certain adverse effects are acceptable is determined based on the severity of the disease. The preparation of a pharmaceutical composition that contains at least one chimeric polypeptide or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

Any reference to nucleotide or amino acid sequences accessible in public sequence databases herein refers to the version of the sequence entry as available on the filing date of this document.

### DESCRIPTION OF EMBODIMENTS

The present invention seeks to provide modified serpin molecules wherein the modifications of residues within the reactive center loop (RCL) increase the specificity for bradykinin-generating proteases of the plasma contact system, particularly at least one of active forms of factor XII (FXII), plasma kallikrein (PK) and plasmin. These modified serpin molecules may be useful in therapy, for example for the treatment of bradykinin-mediated disease.

In a first aspect, the invention relates to a modified serpin molecule wherein one or more of residues of the positions P4, P3, P2, P1, P1′, P2′, P3′ and P4′ in the reactive center loop (RCL) of the serpin are mutated as compared to corresponding unmodified serpin (i.e., the corresponding wild-type serpin without the mutation). Preferably, the invention relates to a modified serpin molecule wherein one or more of residues of the positions P4, P3, P2, P1 and P1′ in the reactive center loop (RCL) of the serpin are mutated as compared to corresponding unmodified serpin (i.e., the corresponding wild-type serpin without the mutation). The mutations preferably increase the inhibition of one or more of proteases selected from PK, plasmin and an active form of FXII, as compared to the corresponding unmodified serpin. The mutation preferably at least increase the inhibition of PK, as compared to the corresponding unmodified (i.e. wild-type) serpin. More preferably, the mutations at least increase the inhibition of PK and plasmin, or the mutations at least increase the inhibition of PK and an active form of FXII, both as compared to the corresponding unmodified serpin. Most preferably, the mutations at least increase the inhibition of all three of PK, plasmin and an active form of FXII, as compared to the corresponding unmodified serpin.

A thus modified serpin of the invention further preferably more strongly inhibits PK than the serpin inhibits thrombin. More preferably the modified serpin also more strongly inhibits PK than the serpin inhibits activated protein C (APC). A preferred modified serpin thus more strongly inhibits PK than the serpin inhibits either one of thrombin or APC.

It is further preferred that a modified serpin of the invention inhibits at least one of PK and an active form of FXII at least as strong as or preferably more strongly than that they are inhibited by C1 esterase inhibitor (C1INH). Preferably, the second-order inactivation rate constant (M⁻¹ s⁻¹) of the modified serpin for PK is increased by at least a factor 1.1, 1.2, 1.5, 2, 3, 4, 5, 10, 20, 50 or 100 as compared to inactivation rate constant of C1 esterase inhibitor for PK, and, the second-order inactivation rate constant of the modified serpin for an active form of FXII is increased by at least a factor 1.1, 1.2, 1.5, 2, 3, 4, 5, 6, 7, 8, 10, 20, 50 or 100 as compared to inactivation rate constant of C1 esterase inhibitor for an active form of FXII. Active forms of FXII are as described below. Second-order inactivation rate constants of serpins can be determined and calculated as e.g., described by Scott et al. (1986 J Clin Invest. 77(2):631-4).

A modified serpin of the invention may have one or more mutations in its reactive center loop (RCL). For example, the modified serpin may have at least one, two, three, four, five, six, seven, eight or nine mutations in its RCL. In particular, the modified serpin may have at least more than four mutations in its RCL. Alternatively, the modified serpin may have at most one, two, three, four, five, six, seven, eight, nine, ten, eleven, twelve, thirteen, fourteen, fifteen, sixteen, seventeen, eighteen, nineteen, or twenty mutations in its RCL.

The modified serpin of the invention preferably that has mutations in one or more of the P4, P3, P2, P1, P1′, P2′, P3′ and P4′ residues of its reactive center loop (RCL). Preferably, the residues at one, two, three or all four of positions P1, P2, P3, and P4 are mutated. For example, the residues at one or both of positions P1 and P2 and optionally P3 and/or P4 may be mutated. Preferably, at least position P1 is mutated, more preferably at least positions P1 and P2 are mutated, and most preferably at least positions P1, P2 and P4 are mutated. The RCL of the modified serpin may thus have mutations at position P1, P2, P3, or P4, at positions P1 and P2, P1 and P3, P1 and P4, P2 and P3, P2 and P4 or P3 and P4, at positions P1, P2 and P3, P1, P2 and P4, P1 P3 and P4 or P2, P3 and P4, or at all four positions P1, P2, P3 and P4. In one embodiment, the one or more mutations in P1, P2, P3 and/or P4 are the only mutations in the RCL of the modified serpin.

In another embodiment, other positions in the RCL may in addition be mutated, such as e.g., one or more of positions P1′, P2′, P3′, and P4′. Preferably therefore, in addition to the above mutations at positions P1-P4, the residues at one, two, three or all four of positions P1′, P2′, P3′, and P4′ are mutated. For example, the residues at one or both of positions P1′ and P2′ and optionally P3′ and/or P4′ may be mutated. Preferably, at least position P1′ is mutated, more preferably at least positions P1′ and P2′ are mutated, and most preferably at least positions P1′, P2′ and P3′ are mutated. The RCL of the modified serpin may thus have mutations at position P1′, P2′, P3′, or P4′, at positions P1′ and P2′, P1′ and P3′, P1′ and P4′, P2′ and P3′, P2′ and P4′ or P3′ and P4′, at positions P1′, P2′ and P3′, P1′, P2′ and P4′, P1′, P3′ and P4′ or P2′, P3′ and P4′, or at all four positions P1′, P2′, P3′ and P4′. The residues at other positions in the RCL may be unmutated wild-type residues.

RCL residues are numbered herein according to the Schechter-Berger nomenclature for substrates and inhibitors of serine proteases (Schechter & Berger 1967). This standard nomenclature allows the residue at specific positions in the RCL, such as positions P4, P3, P2 and/or P1, to be easily identified in any serpin sequence. The reactive center loop (RCL) of a serpin is typically about 27 residues in length and contains the scissile P1-P1′ bond that is cleaved by the target protease (Crowther et al., 1992 Curr Opin Biotechnol August; 3(4):399-407). The RCL extends from strand 5 of beta sheet A to strand 1 of beta sheet C of the serpin. Residues P17 Glu, P15 Gly and P14 Thr are conserved in serpins. For example, the RCL of a serpin may comprise the consensus sequence P17 E, P16 E/K/R, P15 G, P14 T/S, P12-P9 (A/G/S) 4 (Hopkins et al., 1993; Irving et al. 2000). The RCL starts at residue P17 and usually ends at residue P5′. RCLs may be extended in some serpins, such as PCI and α1AT, by additional residues on the P′ side. For example, the RCL of α1-antitrypsin consists of residues P17-P10′ and the RCL of PCI consists of residues P17-P6′.

In one embodiment, the modified serpin comprises an RCL wherein preferably the P1 residue is lysine (K) or arginine (R). The modified serpin further preferably comprises an RCL wherein the P2 residue is not proline (P). A preferred modified serpin comprises an RCL wherein the P4 residue is serine (S). More preferably, the modified serpin comprises an RCL wherein the P1 residue is lysine (K) or arginine (R), wherein the P2 residue is not proline (P) and wherein the P4 residue is serine (S).

In one embodiment, a modified serpin of the invention preferably is a serpin of which the minimal amount of the serpin that inhibits PK activity by at least 50, 55, 60, 65, 70, 75, 80, 85, 90, 92, 94, 95, 96, 97, 98 or 99%, is an amount that inhibits thrombin activity by no more than 15, 12, 10, 7, 6, 5, 4, 3, 2 or 1%. Examples of modified serpins with this inhibition profile include serpins comprising an RCL wherein the residues P4-P1 of the RCL have an amino acid sequence selected from the group consisting of SMTR (SEQ ID NO: 12), SGQR (SEQ ID NO: 13), SVTR (SEQ ID NO: 14), SATR (SEQ ID NO: 15), SFNR (SEQ ID NO: 16), SWKK (SEQ ID NO: 17), SEAR (SEQ ID NO: 18), SVVK (SEQ ID NO: 19), SDYK (SEQ ID NO: 20), SVRK (SEQ ID NO: 21), SPRR (SEQ ID NO: 22), SMDR (SEQ ID NO: 23), SLGR (SEQ ID NO: 24), SKGR (SEQ ID NO: 25), SGNR (SEQ ID NO: 26), and SMHR (SEQ ID NO: 27), as well as serpins comprising an RCL wherein the residues P4-P1 of the RCL have the amino acid sequence SLLR (SEQ ID NO: 28) and wherein the P1′ residue is selected from the group consisting of: Q, F, H, R, K, C, L, Y, N, I, D, W, E, V, M, A, T and P, of which K, L, Y, I, D, E, A, T, M, R and V are preferred, and I and V are most preferred.

In one embodiment, a modified serpin of the invention preferably is a serpin of which the minimal amount of the serpin that inhibits PK activity by at least 50, 55, 60, 65, 70, 75, 80, 85, 90, 92, 94, 95, 96, 97, 98 or 99%, is an amount that inhibits the activities of both thrombin and APC by no more than 15, 12, 10, 7, 6, 5, 4, 3, 2 or 1%. Examples of modified serpins with this inhibition profile include serpins comprising an RCL wherein the residues P4-P1 of the RCL have an amino acid sequence selected from the group consisting of SGQR (SEQ ID NO: 13), SATR (SEQ ID NO: 15), SFNR (SEQ ID NO: 16), SWKK (SEQ ID NO: 17), SEAR (SEQ ID NO: 18), SVVK (SEQ ID NO: 19), SDYK (SEQ ID NO: 20), SVRK (SEQ ID NO: 21), SPRR (SEQ ID NO: 22), SMDR (SEQ ID NO: 2), SLGR (SEQ ID NO: 24), SKGR (SEQ ID NO: 25), and SGNR (SEQ ID NO: 26), as well as serpins comprising an RCL wherein the residues P4-P1 of the RCL have the amino acid sequence SLLR (SEQ ID NO: 28) and wherein the P1′ residue is selected from the group consisting of: Q, F, H, R, K, C, L, Y, N, I, D, W, E, V, M, A, T and P, of which K, L, Y, I, D, E, A, T, M, R and V are preferred, and I and V are most preferred.

In one embodiment, a modified serpin of the invention preferably is a serpin of which the minimal amount of the serpin that inhibits PK activity by at least 50, 55, 60, 65, 70, 75, 80, 85, 90, 92, 94, 95, 96, 97, 98 or 99%, is an amount that inhibits thrombin activity by no more than 15, 12, 10, 7, 6, 5, 4, 3, 2 or 1% and that further inhibits plasmin by at least 15, 19, 20, 30, 35, 40, 45, 50, 55, 60, 65, 70, 75, 80, 85, 90, 92, 94, 95, 96, 97, 98 or 99%. Examples of modified serpins with this inhibition profile include serpins comprising an RCL wherein the residues P4-P1 of the RCL have an amino acid sequence selected from the group consisting of SMTR (SEQ ID NO: 12), SVTR (SEQ ID NO: 14), SWKK (SEQ ID NO: 17), SVVK (SEQ ID NO: 19), SVRK (SEQ ID NO: 21), SPRR (SEQ ID NO: 22), SMDR (SEQ ID NO: 23) and SKGR (SEQ ID NO: 25), as well as serpins comprising an RCL wherein the residues P4-P1 of the RCL have the amino acid sequence SLLR (SEQ ID NO: 28) and wherein the P1′ residue is selected from the group consisting of: Q, F, H, R, K, C, L, Y, N, I, D, W, E, V, M, A, T and P, of which K, L, Y, I, D, E, A, T, M, R and V are preferred, and I and V are most preferred.

In one embodiment, a modified serpin of the invention preferably is a serpin of which the minimal amount of the serpin that inhibits PK activity by at least 50, 55, 60, 65, 70, 75, 80, 85, 90, 92, 94, 95, 96, 97, 98 or 99%, is an amount that inhibits the activities of both thrombin and APC by no more than 15, 12, 10, 7, 6, 5, 4, 3, 2 or 1% and that further inhibits plasmin by at least 15, 19, 20, 30, 35, 40, 45, 50, 55, 60, 65, 70, 75, 80, 85, 90, 92, 94, 95, 96, 97, 98 or 99%. Examples of modified serpins with this inhibition profile include serpins comprising an RCL wherein the residues P4-P1 of the RCL have an amino acid sequence selected from the group consisting of SWKK (SEQ ID NO: 17), SVVK (SEQ ID NO: 19), SVRK (SEQ ID NO: 21), SPRR (SEQ ID NO: 22), SMDR (SEQ ID NO: 23) and SKGR (SEQ ID NO: 25), as well as serpins comprising an RCL wherein the residues P4-P1 of the RCL have the amino acid sequence SLLR (SEQ ID NO: 28) and wherein the P1′ residue is selected from the group consisting of: Q, F, H, R, K, C, L, Y, N, I, D, W, E, V, M, A, T and P, of which K, L, Y, I, D, E, A, T, M, R and V are preferred, and I and V are most preferred.

In one embodiment, a modified serpin of the invention preferably is a serpin of which the minimal amount of the serpin that inhibits PK activity by at least 50, 55, 60, 65, 70, 75, 80, 85, 90, 92, 94, 95, 96, 97, 98 or 99%, is an amount that inhibits thrombin activity by no more than 15, 12, 10, 7, 6, 5, 4, 3, 2 or 1% and that further inhibits an active form of FXII by at least 15, 19, 20, 30, 35, 40, 45, 50, 55, 60, 65, 70, 75, 80, 85, 90, 92, 94, 95, 96, 97, 98 or 99%. Examples of modified serpins with this inhibition profile include serpins comprising an RCL wherein the residues P4-P1 of the RCL have an amino acid sequence selected from the group consisting of SMTR (SEQ ID NO: 12), SVTR (SEQ ID NO: 14), SATR (SEQ ID NO: 15), SLGR (SEQ ID NO: 24) and SKGR (SEQ ID NO: 25), as well as serpins comprising an RCL wherein the residues P4-P1 of the RCL have the amino acid sequence SLLR (SEQ ID NO: 28) and wherein the P1′ residue is selected from the group consisting of: Q, F, H, R, K, C, L, Y, N, I, D, W, E, V, M, A, T and P, of which K, L, Y, I, D, E, A, T, M, R and V are preferred, and I and V are most preferred.

In one embodiment, a modified serpin of the invention preferably is a serpin of which the minimal amount of the serpin that inhibits PK activity by at least 50, 55, 60, 65, 70, 75, 80, 85, 90, 92, 94, 95, 96, 97, 98 or 99%, is an amount that inhibits the activities of both thrombin and APC by no more than 15, 12, 10, 7, 6, 5, 4, 3, 2 or 1% and that further inhibits an active form of FXII by at least 15, 19, 20, 30, 35, 40, 45, 50, 55, 60, 65, 70, 75, 80, 85, 90, 92, 94, 95, 96, 97, 98 or 99%. Examples of modified serpins with this inhibition profile include serpins comprising an RCL wherein the residues P4-P1 of the RCL have an amino acid sequence selected from the group consisting of SATR (SEQ ID NO: 15), SLGR (SEQ ID NO: 24) and SKGR (SEQ ID NO: 25), as well as serpins comprising an RCL wherein the residues P4-P1 of the RCL have the amino acid sequence SLLR (SEQ ID NO: 28) and wherein the P1′ residue is selected from the group consisting of: Q, F, H, R, K, C, L, Y, N, I, D, W, E, V, M, A, T and P, of which K, L, Y, I, D, E, A, T, M, R and V are preferred, and I and V are most preferred.

In a preferred embodiment, a modified serpin of the invention preferably is a serpin of which the minimal amount of the serpin that inhibits PK activity by at least 50, 55, 60, 65, 70, 75, 80, 85, 90, 92, 94, 95, 96, 97, 98 or 99%, is an amount that inhibits thrombin activity by no more than 15, 12, 10, 7, 6, 5, 4, 3, 2 or 1%, that further inhibits plasmin by at least 15, 19, 20, 30, 35, 40, 45, 50, 55, 60, 65, 70, 75, 80, 85, 90, 92, 94, 95, 96, 97, 98 or 99% and that further inhibits an active form of FXII by at least 15, 19, 20, 30, 35, 40, 45, 50, 55, 60, 65, 70, 75, 80, 85, 90, 92, 94, 95, 96, 97, 98 or 99%. Examples of modified serpins with this inhibition profile include serpins comprising an RCL wherein the residues P4-P1 of the RCL have an amino acid sequence selected from the group consisting of SMTR (SEQ ID NO: 12), as well as serpins comprising an RCL wherein the residues P4-P1 of the RCL have the amino acid sequence SLLR (SEQ ID NO: 28) and wherein the P1′ residue is selected from the group consisting of: Q, F, H, R, K, C, L, Y, N, I, D, W, E, V, M, A, T, and P, of which K, L, Y, I, D, E, A, T, M, R and V are preferred, and I and V are most preferred.

In a more preferred embodiment, a modified serpin of the invention preferably is a serpin of which the minimal amount of the serpin that inhibits PK activity by at least 50, 55, 60, 65, 70, 75, 80, 85, 90, 92, 94, 95, 96, 97, 98 or 99%, is an amount that inhibits the activities of both thrombin and APC by no more than 15, 12, 10, 7, 6, 5, 4, 3, 2 or 1%, that further inhibits plasmin by at least 15, 19, 20, 30, 35, 40, 45, 50, 55, 60, 65, 70, 75, 80, 85, 90, 92, 94, 95, 96, 97, 98 or 99% and that further inhibits an active form of FXII by at least 15, 19, 20, 30, 35, 40, 45, 50, 55, 60, 65, 70, 75, 80, 85, 90, 92, 94, 95, 96, 97, 98 or 99%. Examples of modified serpins with this inhibition profile include serpins comprising an RCL wherein the residues P4-P1 of the RCL have the amino acid sequence SLLR (SEQ ID NO: 28) and wherein the P1′ residue is selected from the group consisting of: Q, F, H, R, K, C, L, Y, N, I, D, W, E, V, M, A, T and P, of which K, L, Y, I, D, E, A, T, M, R and V are preferred, and I and V are most preferred.

Preferably, the inhibition of PK activity (e.g., the above percentages inhibition) is determined in an in vitro assay on a chromogenic or fluorogenic PK substrate (e.g., the chromogenic Pro-Phe-Arg-p-nitroanilide peptide substrate L2120), preferably as described in the Examples herein.

Preferably, the inhibition of thrombin activity (e.g., the above percentages inhibition) is determined in an in vitro assay on a chromogenic or fluorogenic thrombin substrate (e.g., the fluorogenic benzyloxycarbonyl-Gly-Gly-Arg-7-amido-4-methylcoumarin peptide substrate 11140), preferably as described in the Examples herein.

Preferably, the inhibition of APC activity (e.g., the above percentages inhibition) is determined in an in vitro assay on a chromogenic or fluorogenic APC substrate (e.g., the chromogenic pyroGlu-Pro-Arg-p-nitroanilide peptide substrate S2366), preferably as described in the Examples herein.

Preferably, the inhibition of active forms of FXII activity (e.g., the above percentages inhibition) is determined in an in vitro assay on a chromogenic or fluorogenic FXII substrate (e.g., the chromogenic Pro-Phe-Arg-p-nitroanilide peptide substrate L2120), preferably as described in the Examples herein.

Preferably, the inhibition of plasmin activity (e.g., the above percentages inhibition) is determined in an in vitro assay on a chromogenic or fluorogenic plasmin substrate (e.g., the fluorogenic Val-Leu-Lys-7-amido-4-methylcoumarin peptide substrate 11390), preferably as described in the Examples herein.

In one embodiment, a modified serpin of the invention further preferably more strongly inhibits one or more of an active form of FXII, PK and plasmin, than the serpin inhibits one or more proteases selected from elastase, chymotrypsin FVIIa, FIXa, and FXa. More preferably, the modified serpin furthermore strongly inhibits one or more of an active form of FXII, PK and plasmin than the serpin inhibits FXIa. Preferably, the serpin is a serpin of which the minimal amount of the serpin that inhibits at least one of the activities of PK, plasmin and an active form of FXII by at least 50, 55, 60, 65, 70, 75, 80, 85, 90, 92, 94, 95, 96, 97, 98 or 99%, is an amount that inhibits at least one of the activities of elastase, chymotrypsin FVIIa, FIXa, FXIa and FXa by no more than 15, 12, 10, 7, 6, 5, 4, 3, 2 or 1%

An active form of FXII is herein understood to include all forms of FXII that have at least some proteolytic activity. Initiation of the contact system of the coagulation cascade requires a small spark of spontaneous enzymatic activity that is generated by FXII. This first pulse of enzymatic activity appears erratic and does not follow the rules of classic enzymatic behavior (Golas et al., 2013, Biomaterials 34, 607-620). This occurs when FXII changes its conformation during its binding to a negatively charged surface, after which it attains a limited amount of proteolytic activity (Ratnoff and Saito, 1979, Proc Natl Acad Sci USA 76, 1461-1463). Furthermore, FXII becomes increasingly susceptible to cleavage by plasma kallikrein (PK) and activated FXII (FXIIa) itself (Griffin, 1978, Proc Natl Acad Sci USA 75, 1998-2002). A single cleavage after an arginine residue at position 353 (R353; counted from the N-terminus in the mature FXII molecule) unlocks the full activity of FXIIa. This cleavage converts FXIIa from a single-chain molecule to a two-chain form, termed α-FXIIa, which is held together by a disulfide bond. This molecular on/off switch of FXII was identified in cross-reactive material positive deficient persons in which this arginine was substituted by a proline residue (Hovinga et al., 1994, Blood 84, 1173-1181). Besides R353, several other protease-sensitive cleavage sites have been identified. Cleavage after an arginine at position 334 separates the surface-binding domains (˜50 kDa) from the active protease domain (˜30 kDa), resulting in fluid-phase activity of FXIIa. This soluble form of FXIIa is termed β-FXIIa or FXIIf. The capacity of FXIIa to cleave more of its own precursor is important to effectively induce contact activation; materials that do not support this reaction, do not trigger contact activation (Citarella et al., 1997, Br J Haematol 99, 197-205). All of these forms of FXII with at least some proteolytic activity are included in the term an active form of FXII. More preferably an active form of FXII includes at least one of α-FXIIa and β-FXIIa or FXIIf.

The modified serpin may show greater inhibition of PK, plasmin and/or an active form of FXII than inhibition of thrombin and one or more of APC, elastase, chymotrypsin FVIIa, FIXa, FXIa and FXa. For example, inhibition of PK, plasmin and/or an active form of FXII by the modified serpin may be 2 fold more, at least 3 fold more, at least 4 fold more, at least 5 fold more, at least 6 fold more, at least 7 fold more, at least 8 fold more, at least 9 fold more at least 10 fold more at least 100 or at least 1000 fold more than inhibition of thrombin, APC, elastase, chymotrypsin FVIIa, FIXa, FXIa and/or FXa by the modified serpin. In some embodiments, the modified serpin may inhibit PK, plasmin and/or an active form of FXII with a second-order rate constant (k2) that is at least 2 fold more, at least 3 fold more, at least 4 fold more, at least 5 fold more, at least 6 fold more, at least 7 fold more, at least 8 fold more, at least 9 fold more, at least 10 fold more at least 100 or at least 1000 fold more than the second-order rate constant for the inhibition of thrombin, APC, elastase, chymotrypsin FVIIa, FIXa, FXIa and/or FXa. Preferably the stoichiometry of inhibition of the modified serpin for PK, plasmin and/or an active form of FXII is 1.

In a preferred embodiment, a modified serpin as described herein may inhibit an active form of one or more of PK, plasmin and/or an active form of FXII but display no inhibition or substantially no inhibition of one or more of thrombin, APC, elastase, activated protein C, FVIIa, FIXa, FXa, FXIa and chymotrypsin. More preferably, the modified serpin display no inhibition or substantially no inhibition of any one of thrombin, APC, elastase, activated protein C, FVIIa, FIXa, FXa and chymotrypsin.

The one or more mutations in the reactive center loop (RCL) of the modified serpin may comprise or consist of a mutation at the P4 position. Preferably, the mutation is a substitution. The native P4 residue in the RCL of the wild-type serpin may be replaced with a non-native residue in the modified serpin. For example, the native Alanine (A) residue at the P4 position in the wild-type sequence of α1-antitrypsin (α1AT) may be replaced with a residue other than A in the modified serpin. Preferably, the P4 residue in the wild-type serpin is replaced with a hydroxylgroup containing residue a small or very small residue, of which a serine (S) residue is most preferred.

The one or more mutations in the reactive center loop (RCL) of the modified serpin may comprise or consist of a mutation at the P3 position. Preferably, the mutation is a substitution. The native P3 residue in the RCL of the wild-type serpin may be replaced with a non-native residue in the modified serpin. For example, the native isoleucine (I) residue at the P3 position in the wild-type serpin may be replaced with a residue other than I in the modified serpin. In some preferred embodiments, the P3 residue in the wild-type serpin is replaced with a hydrophobic residue, an aliphatic residue, a cycloalkenyl-associated residue, a negatively or positively charged residue or an asparagine (N) residue. More preferably, the P3 residue in the wild-type serpin is replaced with an aliphatic residue, of which methionine (M) and leucine (L) are most preferred.

The one or more mutations in the reactive center loop (RCL) of the modified serpin may comprise or consist of a mutation at the P2 position. Preferably, the mutation is a substitution. The native P2 residue in the RCL of the wild-type serpin may be replaced with a non-native residue in the modified serpin. For example, the native P residue at the P2 position in the wild-type sequence of the serpin may be replaced with a residue other than P in the modified serpin. In some preferred embodiments, the P2 residue in the wild-type serpin may be other than P (proline) residue. Preferably, the P3 residue in the wild-type serpin is replaced with a threonine (T) or leucine (L) residue.

The one or more mutations in the reactive center loop (RCL) of the modified serpin may comprise or consist of a mutation at the P1 position. Preferably, the mutation is a substitution. The native P1 residue in the RCL of the wild-type serpin may be replaced with a non-native residue in the modified serpin. For example, the native methionine (M) residue at the P1 position in the wild-type sequence of serpin may be replaced with a residue other than M in the modified serpin. In some preferred embodiments, the P1 residue in the wild-type serpin is replaced with a positively charged residue, of which R (arginine) or K (lysine) residue are more preferred and R (arginine) is most preferred.

Modified serpins where the P4-P1 residues are AIAR are not part of the invention, because contrary to what was disclosed in U.S. Pat. No. 4,973,668, we found that a modified serpin wherein the P4-P1 residues are AIAR does effectively inhibit thrombin.

Preferably, the modified serpin is non-immunogenic in a human. For example, the wild-type serpin may be a human serpin, preferably a human plasma serpin.

One or more residues in the modified serpin may be non-natural amino acids, modified amino acids or D-amino acids. The use of such amino acids is well-known to those of skill in the art.

In some preferred embodiments, the residues in the other positions of the RCL in the serpin may be unmodified i.e., they may be the native residues of the wild-type serpin sequence. The modified serpin may therefore comprise an RCL having a wild-type sequence with mutations at positions P4-P1 as described above.

In some embodiments the modified serpin can further comprise a mutation at the P1′ position. Preferably, the mutation is a substitution. The native P1′ residue in the RCL of the wild-type serpin may be replaced with a non-native residue in the modified serpin. For example, when the modified serpin according to the invention is an α1-antitrypsin (α1AT) serpin, the native S residue at the P1′ position may be replaced with a residue other than S. In some preferred embodiments, the P1′ residue of the modified serpin is replaced with a residue is selected from the group consisting of: Q, F, H, R, K, C, L, Y, N, I, D, W, E, V, M, A, T and P, of which K, L, Y, I, D, E, A, T, M, R and V are preferred, and I and V are most preferred.

In a preferred embodiment, the modified serpin of the invention comprises an RCL wherein the residues P4-P1′ (i.e. P4 to P1 prime) of the RCL have an amino acid sequence selected from the group consisting of SMTRQ (SEQ ID NO: 29), SMTRF (SEQ ID NO: 30), SMTRH (SEQ ID NO: 31), SMTRR (SEQ ID NO: 32), SMTRK (SEQ ID NO: 33), SMTRC (SEQ ID NO: 34), SMTRL (SEQ ID NO: 35), SMTRY (SEQ ID NO: 36), SMTRN (SEQ ID NO: 37), SMTRI (SEQ ID NO: 38), SMTRD (SEQ ID NO: 39), SMTRW (SEQ ID NO: 40), SMTRE (SEQ ID NO: 41), SMTRV (SEQ ID NO: 42), SMTRM (SEQ ID NO: 43), SMTRA (SEQ ID NO: 44), SMTRT (SEQ ID NO: 45), SMTRP (SEQ ID NO: 46), SGQRQ (SEQ ID NO: 47), SGQRF (SEQ ID NO: 48), SGQRH (SEQ ID NO: 49), SGQRR (SEQ ID NO: 50), SGQRK (SEQ ID NO: 51), SGQRC (SEQ ID NO: 52), SGQRL (SEQ ID NO: 53), SGQRY (SEQ ID NO: 54), SGQRN (SEQ ID NO: 55), SGQRI (SEQ ID NO: 56), SGQRD (SEQ ID NO: 57), SGQRW (SEQ ID NO: 58), SGQRE (SEQ ID NO: 59), SGQRV (SEQ ID NO: 60), SGQRM (SEQ ID NO: 61), SGQRA (SEQ ID NO: 62), SGQRT (SEQ ID NO: 63), SGQRP (SEQ ID NO: 64), SVTRQ (SEQ ID NO: 65), SVTRF (SEQ ID NO: 66), SVTRH (SEQ ID NO: 67), SVTRR (SEQ ID NO: 68), SVTRK (SEQ ID NO: 69), SVTRC (SEQ ID NO: 70), SVTRL (SEQ ID NO: 71), SVTRY (SEQ ID NO: 72), SVTRN (SEQ ID NO: 73), SVTRI (SEQ ID NO: 74), SVTRD (SEQ ID NO: 75), SVTRW (SEQ ID NO: 76), SVTRE (SEQ ID NO: 77), SVTRV (SEQ ID NO: 78), SVTRM (SEQ ID NO: 79), SVTRA (SEQ ID NO: 80), SVTRT (SEQ ID NO: 81), SVTRP (SEQ ID NO: 82), SATRQ (SEQ ID NO: 83), SATRF (SEQ ID NO: 84), SATRH (SEQ ID NO: 85), SATRR (SEQ ID NO: 86), SATRK (SEQ ID NO: 87), SATRC (SEQ ID NO: 88), SATRL (SEQ ID NO: 89), SATRY (SEQ ID NO: 90), SATRN (SEQ ID NO: 91), SATRI (SEQ ID NO: 92), SATRD (SEQ ID NO: 93), SATRW (SEQ ID NO: 94), SATRE (SEQ ID NO: 95), SATRV (SEQ ID NO: 96), SATRM (SEQ ID NO: 97), SATRA (SEQ ID NO: 98), SATRT (SEQ ID NO: 99), SATRP (SEQ ID NO: 100), SFNRQ (SEQ ID NO: 101), SFNRF (SEQ ID NO: 102), SFNRH (SEQ ID NO: 103), SFNRR (SEQ ID NO: 104), SFNRK (SEQ ID NO: 105), SFNRC (SEQ ID NO: 106), SFNRL (SEQ ID NO: 107), SFNRY (SEQ ID NO: 108), SFNRN (SEQ ID NO: 109), SFNRI (SEQ ID NO: 110), SFNRD (SEQ ID NO: 111), SFNRW (SEQ ID NO: 112), SFNRE (SEQ ID NO: 113), SFNRV (SEQ ID NO: 114), SFNRM (SEQ ID NO: 115), SFNRA (SEQ ID NO: 116), SFNRT (SEQ ID NO: 117), SFNRP (SEQ ID NO: 118), SWKKQ (SEQ ID NO: 119), SWKKF (SEQ ID NO: 120), SWKKH (SEQ ID NO: 121), SWKKR (SEQ ID NO: 122), SWKKK (SEQ ID NO: 123), SWKKC (SEQ ID NO: 124), SWKKL (SEQ ID NO: 125), SWKKY (SEQ ID NO: 126), SWKKN (SEQ ID NO: 127), SWKKI (SEQ ID NO: 128), SWKKD (SEQ ID NO: 129), SWKKW (SEQ ID NO: 130), SWKKE (SEQ ID NO: 131), SWKKV (SEQ ID NO: 132), SWKKM (SEQ ID NO: 133), SWKKA (SEQ ID NO: 134), SWKKT (SEQ ID NO: 135), SWKKP (SEQ ID NO: 136), SEARQ (SEQ ID NO: 137), SEARF (SEQ ID NO: 138), SEARH (SEQ ID NO: 139), SEARR (SEQ ID NO: 140), SEARK (SEQ ID NO: 141), SEARC (SEQ ID NO: 142), SEARL (SEQ ID NO: 143), SEARY (SEQ ID NO: 144, SEARN (SEQ ID NO: 145), SEARI (SEQ ID NO: 146), SEARD (SEQ ID NO: 147), SEARW (SEQ ID NO: 148), SEARE (SEQ ID NO: 149), SEARV (SEQ ID NO: 150), SEARM (SEQ ID NO: 151), SEARA (SEQ ID NO: 152), SEART (SEQ ID NO: 153), SEARP (SEQ ID NO: 154), SVVKQ (SEQ ID NO: 155), SVVKF (SEQ ID NO: 156), SVVKH (SEQ ID NO: 157), SVVKR (SEQ ID NO: 158), SVVKK (SEQ ID NO: 159), SVVKC (SEQ ID NO: 160), SVVKL (SEQ ID NO: 161), SVVKY (SEQ ID NO: 162), SVVKN (SEQ ID NO: 163), SVVKI (SEQ ID NO: 164), SVVKD (SEQ ID NO: 165), SVVKW (SEQ ID NO: 166), SVVKE (SEQ ID NO: 167), SVVKV (SEQ ID NO: 168), SVVKM (SEQ ID NO: 169), SVVKA (SEQ ID NO: 170), SVVKT (SEQ ID NO: 171), SVVKP (SEQ ID NO: 172), SDYKQ (SEQ ID NO: 173), SDYKF (SEQ ID NO: 174), SDYKH (SEQ ID NO: 175), SDYKR (SEQ ID NO: 176), SDYKK (SEQ ID NO: 177), SDYKC (SEQ ID NO: 178), SDYKL (SEQ ID NO: 179), SDYKY (SEQ ID NO: 180), SDYKN (SEQ ID NO: 181), SDYKI (SEQ ID NO: 182), SDYKD (SEQ ID NO: 183), SDYKW (SEQ ID NO: 184), SDYKE (SEQ ID NO: 185), SDYKV (SEQ ID NO: 186), SDYKM (SEQ ID NO: 187), SDYKA (SEQ ID NO: 188), SDYKT (SEQ ID NO: 189), SDYKP (SEQ ID NO: 190), SVRKQ (SEQ ID NO: 191), SVRKF (SEQ ID NO: 192), SVRKH (SEQ ID NO: 193), SVRKR (SEQ ID NO: 194), SVRKK (SEQ ID NO: 195), SVRKC (SEQ ID NO: 196), SVRKL (SEQ ID NO: 197), SVRKY (SEQ ID NO: 198), SVRKN (SEQ ID NO: 199), SVRKI (SEQ ID NO: 200), SVRKD (SEQ ID NO: 201), SVRKW (SEQ ID NO: 202), SVRKE (SEQ ID NO: 203), SVRKV (SEQ ID NO: 204), SVRKM (SEQ ID NO: 205), SVRKA (SEQ ID NO: 206), SVRKT (SEQ ID NO: 207), SVRKP (SEQ ID NO: 208), SPRRQ (SEQ ID NO: 209), SPRRF (SEQ ID NO: 210), SPRRH (SEQ ID NO: 211), SPRRR (SEQ ID NO: 212), SPRRK (SEQ ID NO: 213), SPRRC (SEQ ID NO: 214), SPRRL (SEQ ID NO: 215), SPRRY (SEQ ID NO: 216), SPRRN (SEQ ID NO: 217), SPRRI (SEQ ID NO: 218), SPRRD (SEQ ID NO: 219), SPRRW (SEQ ID NO: 220), SPRRE (SEQ ID NO: 221), SPRRV (SEQ ID NO: 222), SPRRM (SEQ ID NO: 223), SPRRA (SEQ ID NO: 224), SPRRT (SEQ ID NO: 225), SPRRP (SEQ ID NO: 226), SMDRQ (SEQ ID NO: 227), SMDRF (SEQ ID NO: 228), SMDRH (SEQ ID NO: 229), SMDRR (SEQ ID NO: 230), SMDRK (SEQ ID NO: 231), SMDRC (SEQ ID NO: 232), SMDRL (SEQ ID NO: 233), SMDRY (SEQ ID NO: 234), SMDRN (SEQ ID NO: 235), SMDRI (SEQ ID NO: 236), SMDRD (SEQ ID NO: 237), SMDRW (SEQ ID NO: 238), SMDRE (SEQ ID NO: 239), SMDRV (SEQ ID NO: 240), SMDRM (SEQ ID NO: 241), SMDRA (SEQ ID NO: 242), SMDRT (SEQ ID NO: 243), SMDRP (SEQ ID NO: 244), SLGRQ (SEQ ID NO: 245), SLGRF (SEQ ID NO: 246), SLGRH (SEQ ID NO: 247), SLGRR (SEQ ID NO: 248), SLGRK (SEQ ID NO: 249), SLGRC (SEQ ID NO: 250), SLGRL (SEQ ID NO: 251), SLGRY (SEQ ID NO: 252), SLGRN (SEQ ID NO: 253), SLGRI (SEQ ID NO: 254), SLGRD (SEQ ID NO: 255), SLGRW (SEQ ID NO: 256), SLGRE (SEQ ID NO: 257), SLGRV (SEQ ID NO: 258), SLGRM (SEQ ID NO: 259), SLGRA (SEQ ID NO: 260), SLGRT (SEQ ID NO: 261), SLGRP (SEQ ID NO: 262), SKGRQ (SEQ ID NO: 263), SKGRF (SEQ ID NO: 264), SKGRH (SEQ ID NO: 265), SKGRR (SEQ ID NO: 266), SKGRK (SEQ ID NO: 267), SKGRC (SEQ ID NO: 268), SKGRL (SEQ ID NO: 269), SKGRY (SEQ ID NO: 270), SKGRN (SEQ ID NO: 271), SKGRI (SEQ ID NO: 272), SKGRD (SEQ ID NO: 273), SKGRW (SEQ ID NO: 274), SKGRE (SEQ ID NO: 275), SKGRV (SEQ ID NO: 276), SKGRM (SEQ ID NO: 277), SKGRA (SEQ ID NO: 278), SKGRT (SEQ ID NO: 279), SKGRP (SEQ ID NO: 280), SGNRQ (SEQ ID NO: 281), SGNRF (SEQ ID NO: 282), SGNRH (SEQ ID NO: 283), SGNRR (SEQ ID NO: 284), SGNRK (SEQ ID NO: 285), SGNRC (SEQ ID NO: 286), SGNRL (SEQ ID NO: 287), SGNRY (SEQ ID NO: 288), SGNRN (SEQ ID NO: 289), SGNRI (SEQ ID NO: 290), SGNRD (SEQ ID NO: 291), SGNRW (SEQ ID NO: 292), SGNRE (SEQ ID NO: 293), SGNRV (SEQ ID NO: 294), SGNRM (SEQ ID NO: 295), SGNRA (SEQ ID NO: 296), SGNRT (SEQ ID NO: 297), SGNRP (SEQ ID NO: 298), SMHRQ (SEQ ID NO: 299), SMHRF (SEQ ID NO: 300), SMHRH (SEQ ID NO: 301), SMHRR (SEQ ID NO: 302), SMHRK (SEQ ID NO: 303), SMHRC (SEQ ID NO: 304), SMHRL (SEQ ID NO: 305), SMHRY (SEQ ID NO: 306), SMHRN (SEQ ID NO: 307), SMHRI (SEQ ID NO: 308), SMHRD (SEQ ID NO: 309), SMHRW (SEQ ID NO: 310), SMHRE (SEQ ID NO: 311), SMHRV (SEQ ID NO: 312), SMHRM (SEQ ID NO: 313), SMHRA (SEQ ID NO: 314), SMHRT (SEQ ID NO: 315), SMHRP (SEQ ID NO: 316), SLLRQ (SEQ ID NO: 317), SLLRF (SEQ ID NO: 318), SLLRH (SEQ ID NO: 319), SLLRR (SEQ ID NO: 320), SLLRK (SEQ ID NO: 321), SLLRC (SEQ ID NO: 322), SLLRL (SEQ ID NO: 323), SLLRY (SEQ ID NO: 324), SLLRN (SEQ ID NO: 325), SLLRI (SEQ ID NO: 326), SLLRD (SEQ ID NO: 327), SLLRW (SEQ ID NO: 328), SLLRE (SEQ ID NO: 329), SLLRV (SEQ ID NO: 330), SLLRM (SEQ ID NO: 331), SLLRA (SEQ ID NO: 332), SLLRT (SEQ ID NO: 333) and SLLRP (SEQ ID NO: 334).

A modified serpin as described herein may display the secondary structure of the wild-type serpin, for example a modified serpin may display a structure comprising 3 beta sheets, 8-9 alpha helices and a flexible RCL of about 20 residues.

A modified serpin as described herein may comprise the sequence of a wild-type (i.e., unmodified) serpin, preferably a mature wild-type serpin, with one or more mutations in the RCL thereof as described above, and optionally one or more additional mutations outside the RCL. The sequences of wild-type serpins are well-known in the art and may include as set out herein. The sequences of wild-type serpins may include the sequence of mature wild-type proteins.

The mature α1-antitrypsin (α1AT) (SERPINA1) sequence corresponds to SEQ ID NO: 1. The mature α1-antichymotrypsin (SERPINA3) sequence corresponds to SEQ ID NO: 2. The mature C1-esterase inhibitor (SERPING1) sequence corresponds SEQ ID NO: 3. The mature α2-antiplasmin (SERPINF2) sequence corresponds to SEQ ID NO: 4. The mature antithrombin (ATIII) (SERPINC1) sequence corresponds to SEQ ID NO: 5. The mature heparin cofactor II (HCII) (SERPIND1) sequence corresponds SEQ ID NO: 6. The mature protein C inhibitor (PCI) (SERPINA5) sequence corresponds to SEQ ID NO: 7. The mature kallistatin (SERPINA4) sequence corresponds to SEQ ID NO: 8. The mature plasminogen activator inhibitor (SERPINE1) sequence corresponds to SEQ ID NO: 9. The mature protease nexin 1 (SERPINE2) sequence corresponds to SEQ ID NO: 10. The mature protein Z-dependent protease inhibitor (SERPINA10) sequence corresponds to SEQ ID NO: 11.

Other than mutations of residues in the RCL as described above, a modified serpin may have 50 or fewer amino acid residues altered relative to a wild-type serpin amino acid sequence (for example the mature serpin sequence of one of SEQ ID NO.'s: 1-11, preferably SEQ ID NO: 1), preferably 45 or fewer, 40 or fewer, 30 or fewer, 20 or fewer, 15 or fewer, 10 or fewer, 5 or fewer or 3 or fewer. For example, a modified serpin may comprise the sequence of a wild-type serpin with 50 or fewer, 45 or fewer, 40 or fewer, 30 or fewer, 20 or fewer, 15 or fewer, 10 or fewer, 5 or fewer or 3 or fewer amino acid residues mutated or altered, in addition to the one, two, three, four or five amino acid residues in the RCL of the serpin that are mutated or altered as described above (i.e. the residues at positions P4-P1′).

An amino acid residue in the wild-type amino acid sequence may be altered or mutated by insertion, deletion or substitution, preferably substitution for a different amino acid residue. Such alterations may be caused by one or more of addition, insertion, deletion or substitution of one or more nucleotides in the encoding nucleic acid.

The modified serpin may share at least 50% sequence identity with the wild-type amino acid sequence of a wild-type serpin, for example the mature serpin sequence of any one of SEQ ID NO.'s: 1-11, preferably SEQ ID NO:1, at least 55%, at least 60%, at least 65% at least 70%, at least about 80%, at least 90%, at least 95%, at least 98% or at least 99% sequence identity. For example, a modified serpin may comprise an amino acid sequence having at least 50% sequence identity to residues, whereby the P4-P1′ residues in the RCL are modified as herein described.

In a preferred embodiment, the modified serpin molecule comprises an amino acid sequence that has at least 70%, at least 75%, at least 80%, at least 85%, at least 90%, at least 95%, at least 98%, or at least 99% sequence identity to the sequence of a wild-type serpin, wherein the wild type serpin is selected from the group consisting of α1-antitrypsin (α1AT) (SERPINA1), α1-antichymotrypsin (SERPINA3), C1-esterase inhibitor (SERPING1), α2-antiplasmin (SERPINF2), antithrombin (ATIII) (SERPINC1), heparin cofactor II (HCII) (SERPIND1), protein C inhibitor (PCI) (SERPINA5), Kallistatin (SERPINA4), Plasminogen activator inhibitor (SERPINE1), Protease nexin 1 (SERPINE2) and Protein Z-dependent protease inhibitor (SERPINA10).

α1-antitrypsin (α1AT) (SERPINA1 Gene ID 5265) may have the reference amino acid sequence of NP_000286.3 GI:50363217 (SEQ ID NO: 1) and may be encoded by the reference nucleotide sequence of NM_000295.4 GI:189163524. α1-antichymotrypsin (SERPINA3; Gene ID 12) may have the reference amino acid sequence of NP_001076.2 GI:50659080 (SEQ ID NO: 2) and may be encoded by the reference nucleotide sequence of NM_001085.4 GI:73858562. C1-esterase inhibitor (SERPING1; Gene ID 710) may have the reference amino acid sequence of NP_000053.2 GI: 73858568 (SEQ ID NO: 3) and may be encoded by the reference nucleotide sequence of NM_000062.2GI:73858567. α2-antiplasmin (SERPINF2 Gene ID 5345) may have the reference amino acid sequence of NP_000925.2 encoded by the reference nucleotide sequence of GI:260064047 GI:115583663 (SEQ ID NO: 4) and may be NM 001165920.1. Antithrombin (ATIII) (SERPINC1 Gene ID 462) may have the reference amino acid sequence of NP_000479.1 GI: 4502261 (SEQ ID NO: 5) and may be encoded by the reference nucleotide sequence of NM_000488.3 GI:254588059. Heparin cofactor II (HCII) (SERPIND1 Gene ID3053) may have the reference amino acid sequence of NP_000176.2 GI: 73858566 (SEQ ID NO: 6) and may be encoded by the reference nucleotide sequence of NM 000185.3 GI:73858565. Protein C inhibitor (PCI) (SERPINA5 Gene ID 5104) may have the reference amino acid sequence of NP_000615.3 GI: 194018472 and may (SEQ ID NO: 7) be encoded by the reference nucleotide sequence of NM_000624.5 GI:401782581. Kallistatin (SERPINA4 Gene ID 5267) may have the reference amino acid sequence of NP_006206.2 GI: 21361302 (SEQ ID NO: 8) and may be encoded by the reference nucleotide sequence of NM_006215.2 GI:21361301. Plasminogen activator inhibitor-1 (SERPINE1 Gene ID 5054) may have the reference amino acid sequence of NP_000593.1 GI: 10835159 (SEQ ID NO:9) and may be encoded by the reference nucleotide sequence of NM 000602.4 GI: 383286745. Protease nexin 1 (PNI) (SERPINE2; Gene ID 5270) may have the reference amino acid sequence of NP_001130000.1 GI: 24307907, NP_001130002.1 GI: 211904152 or NP_006207.1 GI: 211904156 (SEQ ID NO: 10) and may be encoded by the reference nucleotide sequence of NM_001136528.1 GI: 211904151, NM_001136530.1 GI: 211904155 or NM 006216.3 GI: 211904150. Protein Z-dependent inhibitor (PZI) (SERPINA10; Gene ID 51156) may have the reference amino acid sequence of NP_057270.1 GI: 7705879 (SEQ ID NO: 11) and may be encoded by the reference nucleotide sequence of NM_016186.2 GI: 154759289.

A modified serpin may further comprise one or more residues that are conserved in wild-type serpin sequences. For example, a modified serpin may comprise some or all of the following residues (numbered according to their position in α1AT): 33F, 49N, 53S, 54P, 56S, 61L, 67G, 72T, 80L, 130F, 147F, 157I, 158N, 161V, 165T, 167G, 169I, 180T, 184L, 186N, 190F, 191K, 192G, 194W, 198F, 203T, 208F, 218V, 220M, 221M, 277Y, 254L, 255P, 289P, 290K, 299L, 303L, 307G, 312F, 316A, 327L, 334H, 342E, 344G, 347A, 369P, 370F, 383L, 384F, 386G, and 391P (Irving et al 2008). The corresponding conserved residues in other serpin sequences may be readily determined using routine sequence analysis.

In another embodiment, the invention relates to a modified serpin molecule wherein the modified serpin molecule is α1AT (SEQ ID NO: 1). Preferably the modified α1AT according to the invention comprises an amino acid sequence that has at least 70% at least 75%, at least 80%, at least 85%, at least 90%, at least 95%, at least 98%, or at least 99% sequence identity to the sequence of a wild-type α1AT, whereby the P4-P1′ residues in the RCL are modified as herein described.

A modified serpin may further comprise modifications in the wild-type sequence that reduce the susceptibility to oxidation. The modified serpin according to the invention may be modified by one or more amino acid substitutions, deletions or insertions to confer reduced susceptibility to oxidation, thereby decreasing degradation of the polypeptide and extending the shelf-life and biological activity of the polypeptide under typical storage, handling and use conditions.

The amino acids that are particularly susceptible to oxidation include methionine (M), cysteine (C), histidine (H), and tyrosine (Y); however, oxidation products have also been observed for proline (P), lysine (K), and arginine (R) (Amici et al., J Biol. Chem. 264:3341-46. 1989; Stadtman, Free Radic Biol Med. 9:315-25, 1990). Amino acid oxidation is typically initiated by the presence of OH- or O₂-reactive species, which may be generated by ionizing radiation (Berlett et al., Proc Natl Acad Sci USA. 1990; 87:389-93). Oxygen reactive species target the protein backbone, stealing a hydrogen atom from an amino acid side group to form a carbon radical. Formation of this carbon radical may ultimately lead to weakened peptide bonds subject to cleavage and protein fragmentation. Oxidation of either methionine (M) 351 or methionine (M) 358 in α1AT has been described to cause loss of its inhibitory activity against elastase (Taggart et al., 2000, J Biol Chem. 275:27258-65). Additionally, it has been described that cysteine (C) 232 in α1AT appears the residue most susceptible to oxidation (Griffiths et al J Biol Chem. 2002 Jul. 12; 277(28):25486-92).

In one embodiment, therefore, one or more of the residues solvent exposed methionine (M) or cysteine (C) residues of the modified serpin according to the invention have been replaced with an amino acid that is not sensitive to oxidation. In the modified serpin of the invention the M (methionine) residue corresponding to the M at position 358 (i.e., position P1 in the RCL) of α1AT is preferably replaced as described herein above.

More preferably, in addition, at least the M (methionine) residue corresponding to the M at position 351 (i.e., position P8 in the RCL) of α1AT is replaced with an I (isoleucine), L (leucine) or V (valine) residue, most preferably a V (valine) residue. Alternatively, or in addition, at least the C (cysteine) residue corresponding to the C at position 232 is replaced, preferably with a serine (S) residue. In a preferred embodiment, the modified serpin (α1AT or other serpin) according to the invention may further comprise modification that reduce the susceptibility to oxidation. The modified serpin according to the invention may be further modified by one or more amino acid substitutions, deletions or insertions to confer reduced susceptibility to oxidation, thereby decreasing degradation of the polypeptide and extending the shelf-life and biological activity of the polypeptide under typical storage, handling and use conditions.

Sequence comparisons are preferably made over the full-length of the relevant sequence described herein.

In a second aspect, the invention relates to a nucleic acid molecule comprising a nucleotide sequence encoding a modified serpin according to the invention.

In a third aspect, the invention relates to a vector comprising a nucleic acid molecule according to the invention. Optionally, the vector according to the invention is a gene therapy vector.

Preferably, the gene therapy vector is a viral gene therapy vector, e.g. a viral gene therapy vector selected from gene therapy vectors based on an adenovirus, an adeno-associated virus (AAV), a herpes virus, a pox virus, an oncolytic virus vector and a retrovirus. A preferred viral gene therapy vector is an AAV or Lentiviral vector.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, lentivirus, and plant viruses), and artificial chromosomes (e.g., YACs). One of skill in the art would be well equipped to construct a vector through standard recombinant techniques, which are described in Sambrook et al, 1989 and Ausubel et al, 1996, both incorporated herein by reference. In addition to encoding a modified polypeptide such as modified gelonin, a vector may encode non-modified polypeptide sequences such as a tag or targeting molecule. A targeting molecule is one that directs the desired nucleic acid to a particular organ, tissue, cell, or other location in a subject's body.

The term “expression vector” refers to a vector containing a nucleic acid sequence coding for at least part of a gene product capable of being transcribed. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described

Techniques for the introduction of nucleic acid into cells are well established in the art and any suitable technique may be employed, in accordance with the particular circumstances. For eukaryotic cells, suitable techniques may include calcium phosphate transfection, DEAE-Dextran, electroporation, liposome-mediated transfection and transduction using retrovirus or other virus, e.g., adenovirus, AAV, lentivirus or vaccinia. For microbial, e.g., bacterial, cells, suitable techniques may include calcium chloride transformation, electroporation and transfection using bacteriophage. Marker genes such as antibiotic resistance or sensitivity genes may be used in identifying clones containing nucleic acid of interest, as is well-known in the art. The introduced nucleic acid may be on an extra-chromosomal vector within the cell or the nucleic acid may be integrated into the genome of the host cell. Integration may be promoted by inclusion of sequences within the nucleic acid or vector which promote recombination with the genome, in accordance with standard techniques. The introduction may be followed by expression of the nucleic acid to produce the encoded modified serpin. In some embodiments, host cells (which may include cells actually transformed although more likely the cells will be descendants of the transformed cells) may be cultured in vitro under conditions for expression of the nucleic acid, so that the encoded serpin polypeptide is produced, inducible promoter is used, expression may require the activation of the inducible promoter.

In a fourth aspect, the invention relates to a host cell comprising a vector according to the invention, which host cell expresses a modified serpin according to invention.

The cell preferably is an isolated cell or a cultured cell. Among the host cells that may be employed are prokaryotes, yeast or higher eukaryotic cells. Prokaryotes include gram negative or gram-positive organisms, for example Escherichia coli or bacilli. Higher eukaryotic cells include insect cells and established cell lines of mammalian origin. Examples of suitable mammalian host cell lines include the COS-7 line of monkey kidney cells (Gluzman et al., 1981, Cell 23:175), L cells, 293 cells, C127 cells, 3T3 cells, Chinese hamster ovary (CHO) cells, HeLa cells, BHK cell lines, and the CVI/EBNA cell line derived from the African green monkey kidney cell line CVI as described by McMahan et al., 1991, EMBO J. 10: 2821. Appropriate cloning and expression vectors for use with bacterial, fungal, yeast, and mammalian cellular hosts are described by Pouwels et al. (Cloning Vectors: A Laboratory Manual, Elsevier, New York, 1985).

The transformed cells can be cultured under conditions that promote which host cell expresses a modified serpin according to invention. Thus, in one aspect the invention relates to a method for producing a modified serpin according to invention, the method comprising the step of cultivating a cell comprising at least one expression vector as defined herein, under conditions conducive to expression of a modified serpin according to invention, optionally, recovering the modified serpin according to invention.

A modified serpin according to invention can be recovered by conventional protein purification procedures, including e.g., protein A-Sepharose, hydroxylapatite chromatography, gel electrophoresis, dialysis, or affinity chromatography (see e.g., Low et al., 2007, J. Chromatography B, 848:48-63; Shukla et al., 2007, J. Chromatography B, 848:28-39

In a fifth aspect, the invention relates to a pharmaceutical composition comprising and/or consisting of a modified serpin according to the invention, a nucleic acid according to the invention, a vector or gene therapy vector according to the invention, or a host cell according to the invention and a pharmaceutically acceptable excipient.

The pharmaceutical composition further preferably comprises at least one pharmaceutically acceptable carrier. The pharmaceutically acceptable carrier such as an adjuvant, or vehicle, is for administration of the antibody or antibody fragment to a subject. Said pharmaceutical composition can be used in the methods of treatment described herein below by administration of an effective amount of the composition to a subject in need thereof. The term “subject”, as used herein, refers to all animals classified as mammals and includes, but is not restricted to, primates and humans. The subject is preferably a male or female human of any age or race.

The term “pharmaceutically acceptable carrier”, as used herein, is intended to include any and all solvents, dispersion media, coatings, antibacterial and antifungal agents, isotonic and absorption delaying agents, and the like, compatible with pharmaceutical administration (see e.g., “Handbook of Pharmaceutical Excipients”, Rowe et al eds. 7^{th} edition, 2012, on the World-Wide Web at pharmpress.com). The use of such media and agents for pharmaceutically active substances is well known in the art. Except insofar as any conventional media or agent is incompatible with the active compound, use thereof in the compositions is contemplated. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter ions such as sodium; metal complexes (e.g. Zn²⁺⁻protein complexes); and/or non-ionic surfactants such as TWEEN™, PLURONICS™ or polyethylene glycol (PEG).

Supplementary active compounds can also be incorporated into the pharmaceutical composition of the invention. Thus, in a particular embodiment, the pharmaceutical composition of the invention may also contain more than one active compound as necessary for the particular indication being treated, preferably those with complementary activities that do not adversely affect each other. For example, it may be desirable to further provide a chemotherapeutic agent, a cytokine, an analgesic agent, or an immunomodulating agent, e.g., an immunosuppressive agent or an immunostimulating agent. The effective amount of such other active agents depends, among other things, on the amount of antibody of the invention present in the pharmaceutical composition, the type of disease or disorder or treatment, etc.

In an embodiment, the modified serpin according to the invention is prepared with carriers that will protect said compound against rapid elimination from the body, such as a controlled release formulation, including implants and microencapsulated delivery systems, e.g., liposomes. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions, including targeted liposomes can also be used as pharmaceutically acceptable carriers. These can be prepared according to methods known to those skilled in the art, for example, as described in U.S. Pat. No. 4,522,811 or WO2010/095940.

The administration route of the modified serpin according to the invention can be parenteral. The term “parenteral” as used herein includes intravenous, intra-arterial, intralymphatic, intraperitoneal, intramuscular, subcutaneous, rectal or vaginal administration. The intravenous forms of parenteral administration are preferred. By “systemic administration” is meant oral, intravenous, intraperitoneal and intramuscular administration. The amount of the modified serpin required for therapeutic or prophylactic effect will, of course, vary with the modified serpin chosen, the nature and severity of the condition being treated and the patient. In addition, the modified serpin may suitably be administered by pulse infusion, e.g., with declining doses of the modified serpin. Preferably the dosing is given by injections, most preferably intravenous or subcutaneous injections, depending in part on whether the administration is brief or chronic.

Thus, in a particular embodiment, the pharmaceutical composition of the invention may be in a form suitable for parenteral administration, such as sterile solutions, suspensions or lyophilized products in the appropriate unit dosage form. Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, CremophorEM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by use of a coating such as lecithin, by the maintenance of the required particle size in the case of dispersion and by use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol or sodium chloride in the composition.

Prolonged absorption of the injectable compositions can be brought about by including in the composition an agent which delays absorption, for example, aluminium monostearate and gelatin.

Sterile injectable solutions can be prepared by incorporating the active compound (e.g a modified serpin) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In a particular embodiment, said pharmaceutical composition is administered via intravenous (IV) or subcutaneous (SC). Adequate excipients can be used, such as bulking agents, buffering agents or surfactants. The mentioned formulations will be prepared using standard methods for preparing parenterally administrable compositions as are well known in the art and described in more detail in various sources, including, for example, “Remington: The Science and Practice of Pharmacy” (Ed. Allen, L. V. 22nd edition, 2012, on the World-Wide Web at pharmpress.com).

It is especially advantageous to formulate the pharmaceutical compositions, namely parenteral compositions, in dosage unit form for ease administration and uniformity of dosage. Dosage unit form as used herein refers to physically discrete units suited as unitary dosages for the subject to be treated; each unit containing a predetermined quantity of active compound (antibody of the invention) calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Generally, an effective administered amount of a modified serpin according to the invention will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.001 to 1,000 mg/kg body weight/day, preferably about 0.01 to about 100 mg/kg body weight/day, most preferably from about 0.05 to 10 mg/kg body weight/day.

Aside from administration of a modified serpin according to the invention to the patient, the present application contemplates administration of a modified serpins by gene therapy. WO96/07321 relates the use of gene therapy to generate.

The pharmaceutical compositions can be included in a container, pack, or dispenser together with instructions for administration.

The antibodies and pharmaceutical compositions of this invention may be used with other drugs to provide a combination therapy. The other drugs may form part of the same composition or be provided as a separate composition for administration at the same time or at different time.

In a sixth aspect, the invention pertains to a modified serpin molecule as described herein for use in the treatment or prevention of a bradykinin-mediated disease. In one embodiment, the modified serpin according to the invention may be used in the treatment or prevention of a bradykinin-mediated disease wherein the bradykinin-mediated disease is selected from the group consisting of non-histamine-dependent idiopathic angioedema, rheumatoid arthritis, Crohn's disease, ulcerative colitis, lupus, Alzheimer's disease, septic shock, burn injury, brain ischemia/reperfusion injury, cerebral edema, diabetic retinopathy, diabetic nephropathy, macular edema, vasculitis, arterial or venous thrombosis, thrombosis associated with ventricular assist devices or stents, heparin-induced thrombocytopenia with thrombosis, thromboembolic disease, and coronary heart disease with unstable angina pectoris, edema, eye disease, gout, intestinal bowel disease, oral mucositis, neuropathic pain, inflammatory pain, spinal stenosis degenerative spine disease, post-operative ileus, aortic aneurysm, osteoarthritis, hereditary angioedema, pulmonary embolism, stroke, head trauma or peri-tumor brain edema, sepsis, acute middle cerebral artery (MCA) ischemic event (stroke), restenosis (e.g., after angioplasty), systemic lupus erythematosis nephritis, an autoimmune disease, an inflammatory disease for example (chronic) auto-inflammatory urticaria, a cardiovascular disease, a neurological disease, a disease associated with protein misfolding, a disease associated with angiogenesis, hypertensive nephropathy and diabetic nephropathy, allergic and respiratory diseases (e.g. anaphylaxis, asthma, chronic obstructive pulmonary disease, acute respiratory distress syndrome, cystic fibrosis, persistent, rhinitis, urticarial and histamine dependent angioedema), tissue injuries (e.g. burn or chemical injury), and poli-trauma (e.g. car crashed and battle injuries).

In a seventh aspect, the invention relates to a gene therapy vector comprising a nucleotide sequence encoding a modified serpin according to the invention for use in the treatment or prevention of a bradykinin-mediated disease. Suitable gene therapy vectors are known per se to the skilled person and include e.g., the viral vectors derived from viruses such as including retrovirus, adenovirus, lentivirus, herpes simplex, vaccinia and adeno-associated virus, as well as non-viral vectors. Preferably, in the gene therapy vector the nucleotide sequence encoding a modified serpin is operably linked to a promoter that ensures expression of the coding sequence in hepatocytes. In one embodiment, the gene therapy vector comprising a nucleotide sequence encoding a modified serpin according is used in the treatment or prevention of a bradykinin-mediated disease wherein the bradykinin-mediated disease is selected from the group consisting of hereditary angioedema, idiopathic angioedema, rheumatoid arthritis, Crohn's disease, ulcerative colitis, lupus, Alzheimer's disease, septic shock, burn injury, brain ischemia/reperfusion injury, cerebral edema, diabetic retinopathy, diabetic nephropathy, macular edema, vasculitis, arterial or venous thrombosis, thrombosis associated with ventricular assist devices or stents, heparin-induced thrombocytopenia with thrombosis, thromboembolic disease, and coronary heart disease with unstable angina pectoris, edema, eye disease, gout, intestinal bowel disease, oral mucositis, neuropathic pain, inflammatory pain, spinal stenosis-degenerative spine disease, post-operative ileus, aortic aneurysm, osteoarthritis, hereditary angioedema, pulmonary embolism, stroke, head trauma or peri-tumor brain edema, sepsis, acute middle cerebral artery (MCA) ischemic event (stroke), restenosis (e.g., after angioplasty), systemic lupus erythematosis nephritis, an autoimmune disease, an inflammatory disease for example (chronic) auto-inflammatory urticaria, a cardiovascular disease, a neurological disease, a disease associated with protein misfolding, a disease associated with angiogenesis, hypertensive nephropathy and diabetic nephropathy, allergic and respiratory diseases (e.g. anaphylaxis, asthma, chronic obstructive pulmonary disease, acute respiratory distress syndrome, cystic fibrosis, persistent, rhinitis, urticarial and histamine dependent angioedema), tissue injuries (e.g. burn or chemical injury) and poli-trauma (e.g. car crashed and battle injuries).

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such these are within the scope of the present invention.

In this document and in its claims, the verb “to comprise” and its conjugations is used in its non-limiting sense to mean that items following the word are included, but items not specifically mentioned are not excluded. Other aspects and embodiments of the invention provide the aspects and embodiments described above with the term “comprising” replaced by the term “consisting of” and the aspects and embodiments described above with the term “comprising” replaced by the term “consisting essentially of”.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

All documents and sequence database entries mentioned in this specification are incorporated herein by reference in their entirety for all purposes.

The present invention has been described above with reference to a number of exemplary embodiments as shown in the drawings. Modifications and alternative implementations of some parts or elements are possible and are included in the scope of protection as defined in the appended claims.

### DESCRIPTION OF THE FIGURES

FIG. 1. Inhibition of plasma kallikrein-like activity in human plasma by various α1-Antitrypsin variants as indicated compared to inhibition by plasma-derived C1 esterase inhibitor. Plasma kallikrein-like activity activity is determined using the chromogenic substrate L2120.

FIG. 2. Inhibition of plasma kallikrein-like activity in mouse plasma by various α1-Antitrypsin variants as indicated. Plasma kallikrein-like activity activity is determined using the chromogenic substrate L2120.

FIG. 3. Inhibition of Factor XIIa activity by α1-Antitrypsin Pittsburgh variants wherein the P1 prime (P1′) position was altered to every natural occuring amino acid as indicated.

FIG. 4. Inhibition of plasma kallikrein activity by α1-Antitrypsin Pittsburgh variants wherein the P1 prime (P1′) position was altered to every natural occuring amino acid as indicated.

FIG. 5. Inhibition of plasmin activity by α1-Antitrypsin Pittsburgh variants wherein the P1 prime (P1′) position was altered to every natural occuring amino acid as indicated.

FIG. 6. Inhibition of thrombin activity by α1-Antitrypsin Pittsburgh variants wherein the P1 prime (P1′) position was altered to every natural occuring amino acid as indicated.

FIG. 7. Inhibition of activated protein C activity by α1-Antitrypsin Pittsburgh variants wherein the P1 prime (P1′) position was altered to every natural occuring amino acid as indicated.

### EXAMPLES

### Example 1

Modified Serpin Construction

The SERPINA1 cDNA sequence was obtained from the NCBI database (NM_001127707.1). The sequence coding for the signal peptide was discarded and replaced by a sequence coding for a Tobacco Etch Virus cleavage site. At the 5′ side an EcoRI digestion site was added and at the 3′ side and NotI digestion was added after the STOP codon of SERPINA1. Hereafter the ³⁵⁴AIPM³⁵⁸ sequence (amino acid numbering without the signal peptide) was replaced with SMTR sequence (Codon sequence was grafted from wild type F12 NCBI: NM_000505.3). The construct was obtained from IDT (Integrated DNA Technologies, Leuven, Belgium) as a custom gene construct. The custom gene construct was propagated in top E. coli TOP10 and selected by ampicillin resistance. Obtained plasmid DNA was digested by EcoRI and NotI. The resulting insert (1223 bp) was separated on and isolated from agarose gel and ligated into a modified pcDNA6 expression vector (pSM2) (De Maat et al, 2016 Clin Immunol November; 30; 138(5):1414-23)). pSM2 encodes for a N-terminal murine IgK secretion signal and a double STREP isolation tag where after the SerpinA1 construct is ligated.

For the construction of the wild type SERPINA1 or the other mutants the C-terminal part of the SERPINA1 sequence was replaced via BsteII-NotI digestion. The new constructs were ordered from IDT as gene block and ligated in to the digested SERPINA1-³⁵⁴SMTR³⁵⁸-pSM2 backbone.

### Example 2

α1-Antitrypsin Expression

The SERPINA1-pSM2 constructs were transfected into HEK293 FreeStyle™ cells using 239Fectin as instructed by the manufacturer (ThermoFisher). After 4 days of protein production, the cells were spun down at 2000×g for 5 minutes. Hereafter the supernatant was collected and stored at −20° C. until further use.

### Example 3

Enzyme Inhibition by α1-Antitrypsin Variants

10 μl of supernatant containing the α1-Antitrypsin variants was incubated with 10 μl of enzyme and 20 μl of buffer (0.2% w/v bovine serum albumin in Hepes buffered saline: 10 mM HEPES, 150 mM NaCl, pH=7.4). After a 5-minute incubation, the substrate was added and substrate conversion was monitored according to substrate specifications. Inhibition percentage was determined within the linear part of the substrate conversion, where substrate conversion without the presence of any α1-AT variant was set as 0% inhibition.

Enzymes
Human αFXIIa (final concentration: 2 μg/mL)
Human βFXIIa (final concentration: 0.7 μg/mL)
Plasma kallikrein (final concentration: 2 μg/mL)
Thrombin (final concentration: 2 U/mL)
Plasmin (final concentration: 2 μg/mL)
Activated protein C (final concentration: 2 μg/mL)

Substrates

| | | Final | | |
|---|---|---|---|---|
| Enzyme | Substrate | Concentration | Method | Wavelength |
| FXIIa | L2120 | 0.5 mM | Absorption | 405 nm |
| PK | L2120 | 0.5 mM | Absorption | 405 nm |
| Thrombin | I1140 | 2 mM | Fluorescence | Ext: 380 Em: 460 |
| Plasmin | I1390 | 0.5 mM | Fluorescence | Ext: 380 Em: 460 |
| APC | S2366 | 0.5 mM | Absorption | 405 nm |

The following substrates where used:
L2120 (Bachem, Cat #L-2120.0100), H-D-Pro-Phe-Arg-pNA.2 HCl; a chromogenic substrate for the determination of plasma kallikrein-like activity used as described in de Maat et al. (2016 Clin Immunol November; 30; 138(5):1414-23).
I1140 (Bachem, Cat #1-1140.0100), Benzyloxycarbonyl-Gly-Gly-Arg-7-amido-4-methylcoumarin.HCl; can used as substrate in a direct fluorometric assay of urokinase and tissue-type plasminogen activator as well as for assaying trypsin and thrombin used as described in Chowdary et al. (2015, Br. J. Haematol., 168: 719).
I1390 (Bachem, Cat #1-1390.0050), H-D-Val-Leu-Lys-7-amido-4-methylcoumarin acetate salt; a sensitive, highly specific fluorescent substrate for plasmin used as described in de Maat et al. (2016 Clin Immunol November; 30; 138(5):1414-23).
S2366 (Chromogenix; Cat #S2366), pyroGlu-Pro-Arg-pNA·HCl; Chromogenic substrate for activated protein C and factor XIa as described in Hubbard et al. (1988, Thromb Haemost; 59, 464-467).

Results of the inhibition of the relevant proteases by the various α1AT variants are provide in Table 1.

TABLE 1
Overview of the percentage inhibition of the relevant proteases by the various α1-Antitrypsin variants of the invention.

| | | | | | | | | | | | | | | Plasma | | | | | | | |
|---|---|---|---|---|---|---|---|---|---|---|---|---|---|---|---|---|---|---|---|---|---|
| | Amino acid residues in RCL positions | | | | | | | | | αFXIIa | | βFXIIa | | Kallikrein | | Plasmin | | Thrombin | | APC | |
| α1-AT | P8 and P4-P4′ | | | | | | | | | Aver- | St | Aver- | St | Aver- | St | Aver- | St | Aver- | St | Aver- | St |
| variants | P8 | P4 | P3 | P2 | P1 | P1′ | P2′ | P3′ | P4′ | age | Dev | age | Dev | age | Dev | age | Dev | age | Dev | age | Dev |
| WT | M | A | I | P | M | S | I | P | P | 0.0 | 0.0 | 0.0 | 0.0 | 0.0 | 0.0 | 0.0 | 0.0 | 0.0 | 0.0 | 0.0 | 0.0 |
| M358R | M | A | I | P | R | S | I | P | P | 94.0 | 2.8 | 90.0 | — | 91.5 | 3.5 | 100.0 | 0.0 | 100.0 | 0.0 | 94.0 | 4.2 |
| 357AR358 | M | A | I | A | R | S | I | P | P | 62.5 | 10.6 | 49.0 | — | 98.0 | 0.0 | 99.0 | 1.4 | 97.5 | 0.7 | 66.5 | 17.7 |
| SMTR | M | S | M | T | R | S | I | P | P | 88.5 | 9.2 | 85.0 | — | 98.5 | 0.7 | 94.0 | 1.4 | 10.5 | 0.7 | 75.0 | 18.4 |
| SMTRox* | V | S | M | T | R | S | I | P | P | 92.5 | 3.5 | 89.2 | 4.8 | 98.7 | 0.8 | 94.5 | 1.4 | 10.3 | 1.5 | 76.5 | 8.4 |
| SMTRox*- | V | S | M | T | R | V | V | G | G | NT | — | 29.0 | — | 47.0 | — | 8.0 | — | 1.0 | — | 0.0 | — |
| VVGG | | | | | | | | | | | | | | | | | | | | | |
| SMTRox*- | V | S | M | T | R | G | V | G | G | NT | — | 9.0 | — | 39.0 | — | 20.0 | — | 3.0 | — | 3.0 | — |
| GVGG | | | | | | | | | | | | | | | | | | | | | |
| SGQRox* | V | S | G | Q | R | S | I | P | P | 4.5 | 2.1 | 4.0 | — | 88.0 | 1.4 | 5.0 | 7.1 | 14.5 | 0.7 | 6.5 | 0.7 |
| SVTRox* | V | S | V | T | R | S | I | P | P | 47.0 | — | 39.0 | — | 99.0 | — | 97.0 | — | 7.0 | — | 49.0 | — |
| SATRox* | V | S | A | T | R | S | I | P | P | 17.0 | — | 15.0 | — | 94.0 | — | 0.0 | — | 3.0 | — | 10.0 | — |
| SFNRox* | V | S | F | N | R | S | I | P | P | 5.0 | — | 3.0 | — | 94.0 | — | 3.0 | — | 7.0 | — | 5.0 | — |
| SWKKox* | V | S | W | K | K | S | I | P | P | NT | — | 5.0 | — | 94.0 | — | 47.0 | — | 0.0 | — | 0.0 | — |
| SEARox* | V | S | E | A | R | S | I | P | P | NT | — | 6.0 | — | 92.0 | — | 13.0 | — | 0.0 | — | 3.0 | — |
| SLLRox* | V | S | L | L | R | S | I | P | P | NT | — | 41.0 | 3.5 | 97.7 | 1.5 | 94.7 | 1.5 | 62.3 | 8.1 | 9.7 | 5.0 |
| SLLRox*-V | V | S | L | L | R | V | I | P | P | NT | — | 47.0 | 8.5 | 97.5 | 0.7 | 68.0 | 1.4 | 4.5 | 4.9 | 3.0 | 2.8 |
| SLLRox*-I | V | S | L | L | R | I | I | P | P | NT | — | 40.3 | 8.5 | 91.3 | 5.0 | 66.7 | 2.9 | 3.7 | 3.2 | 2.3 | 2.1 |
| SVVKox* | V | S | V | V | K | S | I | P | P | 4.0 | — | 2.0 | — | 55.0 | — | 95.0 | — | 6.0 | — | 8.0 | — |
| SDYKox* | V | S | D | Y | K | S | I | P | P | NT | — | 7.0 | — | 50.0 | — | 7.0 | — | 0.0 | — | 1.0 | — |
| SVRKox* | V | S | V | R | K | S | I | P | P | 2.0 | — | 0.0 | — | 91.0 | — | 97.0 | — | 1.0 | — | 4.0 | — |
| SPRRox* | V | S | P | R | R | S | I | P | P | 1.0 | — | 2.0 | — | 97.0 | — | 19.0 | — | 0.0 | — | 1.0 | — |
| SMDRox* | V | S | M | D | R | S | I | P | P | NT | — | 4.0 | — | 87.0 | — | 20.0 | — | 0.0 | — | 0.0 | — |
| SLGRox* | V | S | L | G | R | S | I | P | P | NT | — | 17.3 | 2.9 | 95.5 | 0.6 | 5.3 | 2.4 | 9.5 | 2.1 | 1.0 | 1.2 |
| SKGRox* | V | S | K | G | R | S | I | P | P | NT | — | 22.0 | — | 97.0 | — | 37.0 | — | 7.0 | — | 0.0 | — |
| SGNRox* | V | S | G | W | R | S | I | P | P | NT | — | 5.2 | 1.3 | 71.6 | 1.9 | 1.0 | 2.2 | 0.0 | 0.0 | 1.0 | 1.0 |
| SYARox* | V | S | Y | A | R | S | I | P | P | NT | — | 20.0 | — | 97.0 | — | 0.0 | — | 62.0 | — | 7.0 | — |
| SMHRox* | V | S | M | H | R | S | I | P | P | NT | — | 18.3 | 4.0 | 96.3 | 0.6 | 73.0 | 6.1 | 10.0 | 1.0 | 34.0 | 5.6 |
| SNSRox* | V | S | N | S | R | S | I | P | P | NT | — | 20.0 | — | 97.0 | — | 0.0 | — | 62.0 | — | 7.0 | — |

*“ox” indicates that methionine (M) residues 351 (P8) is replaced with a serine (S) residues to prevent oxidative inactivation of α1AT

### Example 4

Inhibition in Human and in Mouse Plasma

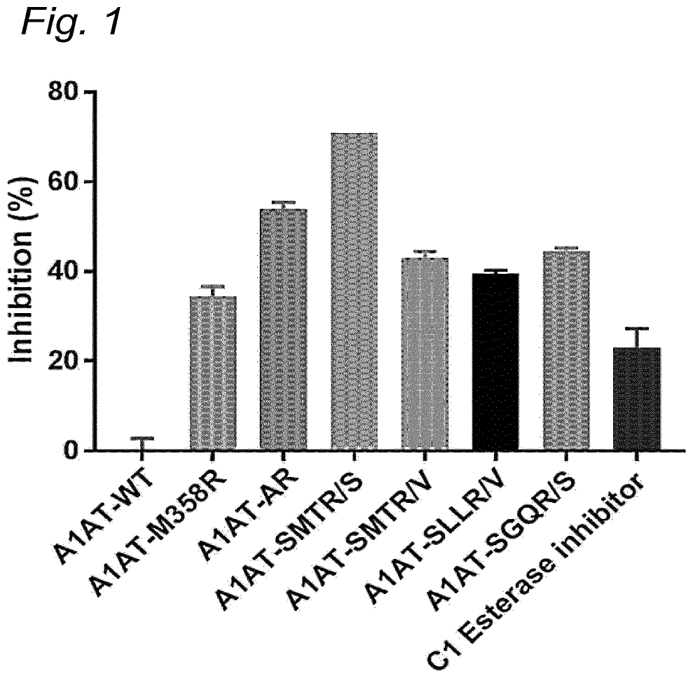

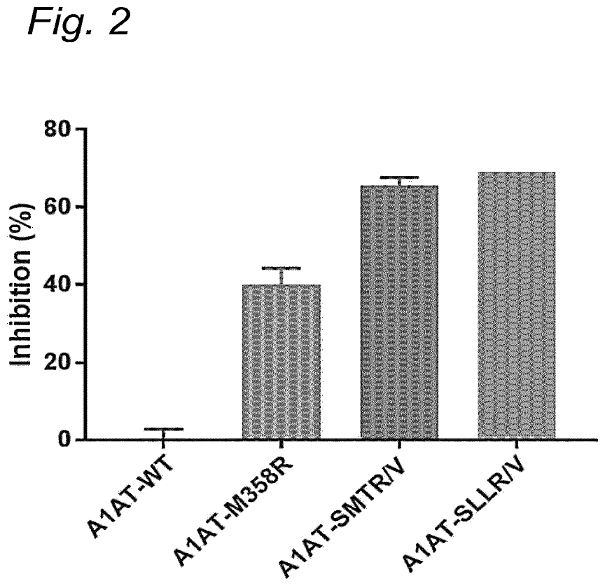

α1-Antitrypsin variants as indicated in FIGS. 1 and 2 were tested for their ability to inhibit plasma kallikrein-like activity as determined with the chromogenic substrate L2120 in respectively human and mouse plasma. In human plasma the inhibition by α1-Antitrypsin variants was also compared with that by plasma-derived C1 esterase inhibitor (Alpha Diagnostics; C1 E15-N).

50 μl of human plasma (3.2% Citrate) was mixed with 20 μl α1-Antitrypsin variant (20 μg/mL final conc.) or C1 esterase inhibitor (40 μg/mL final conc.), 20 μl L2120 chromogenic PK substrate (0.5 mM final conc.) and 10 μl Kaolin (18.75 μg/mL final conc.). Inhibition of plasma kallikrein-like activity was determined as described in Example 3. Results are shown in FIG. 1.

20 μl of mouse plasma (3.2% Citrate) was mixed with 20 μl α1-Antitrypsin variant (100 μg/mL final conc.), 10 μl L2120 chromogenic PK substrate (0.5 mM final conc.) and 5 μl Kaolin (135 μg/mL final conc.). Inhibition of plasma kallikrein-like activity was determined as described in Example 3. Results are shown in FIG. 2.

### Example 5

Enzyme Inhibition by α1-Antitrypsin P1 Prime-Variants

The amino acid of the P1 prime (P1′) position was altered to every natural occurring amino acid. As a template sequence we used the α1-Antitrypsin-Pittsburgh sequence having the P4-P3-P2-P1/P1′-P2′-P3′-P4 sequence′: AIPR/XIPP for the library, wherein X marks the P1′ position in which the amino acid was varied.

α1-Antitrypsin variants were produced as described above in Example 2, except that supernatant was harvested after 5 days. Empty production vector was used as a negative control (No A1AT). Production of the α1-Antitrypsin variants was quantified by Coomassie Blue and supernatants were diluted in pSM2 media to equalize the concentrations of the different α1AT variants. Enzyme inhibition by the α1-Antitrypsin P1′-variants was tested as described in Example 3 above.

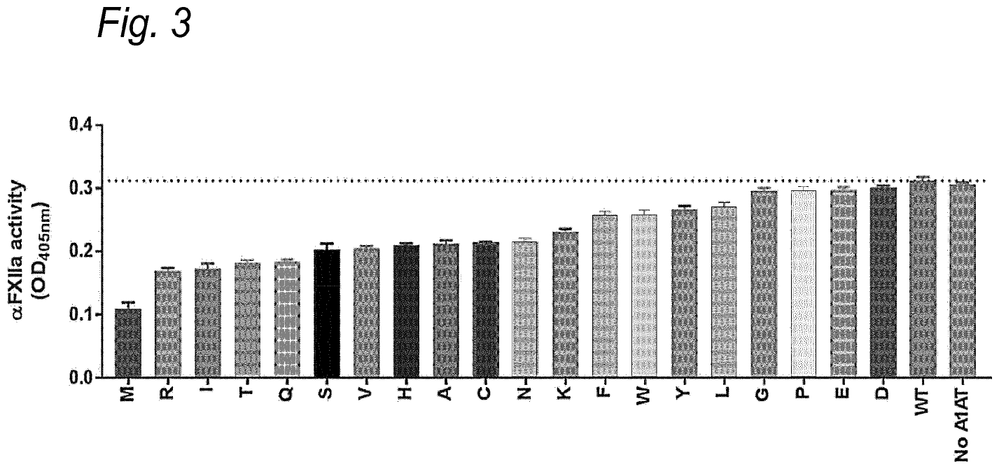

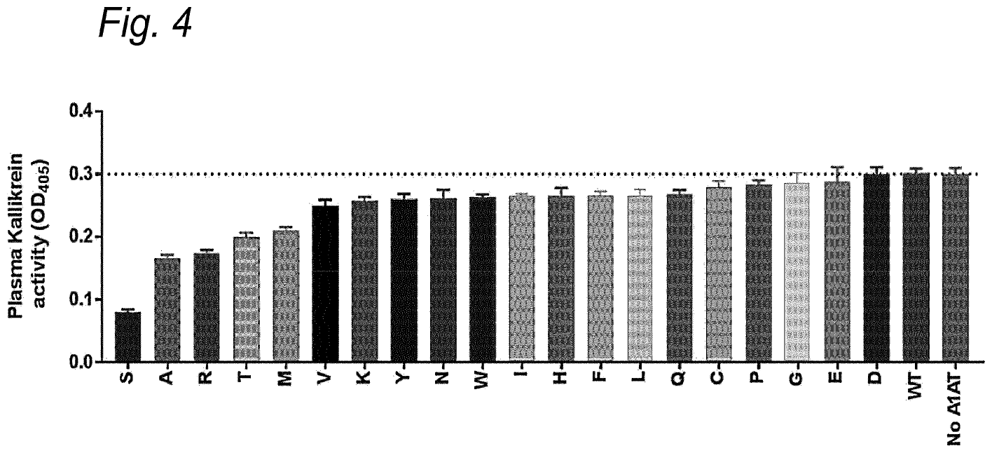

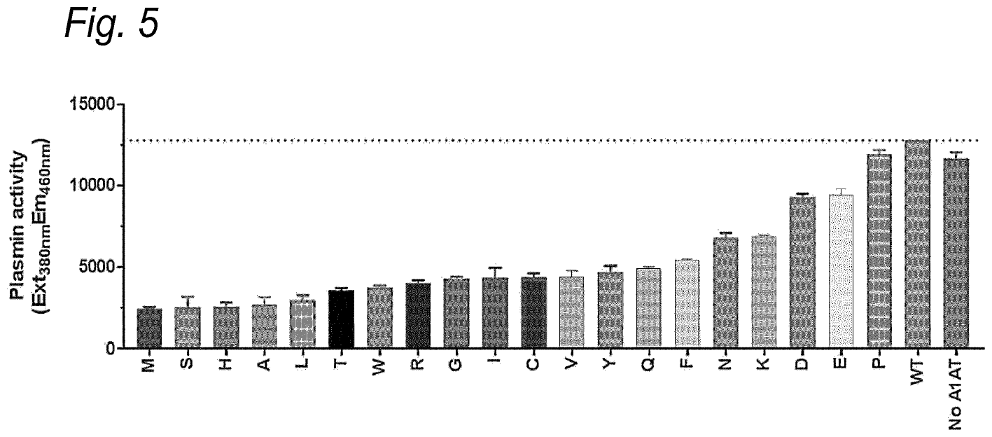

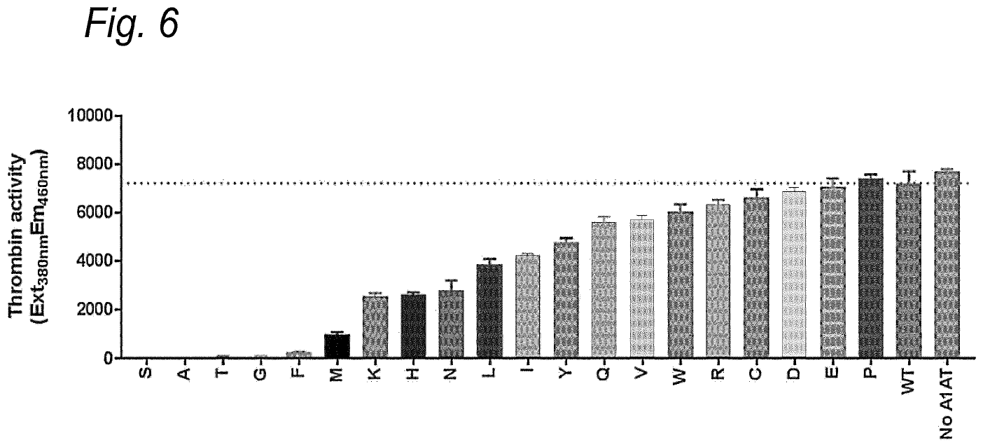

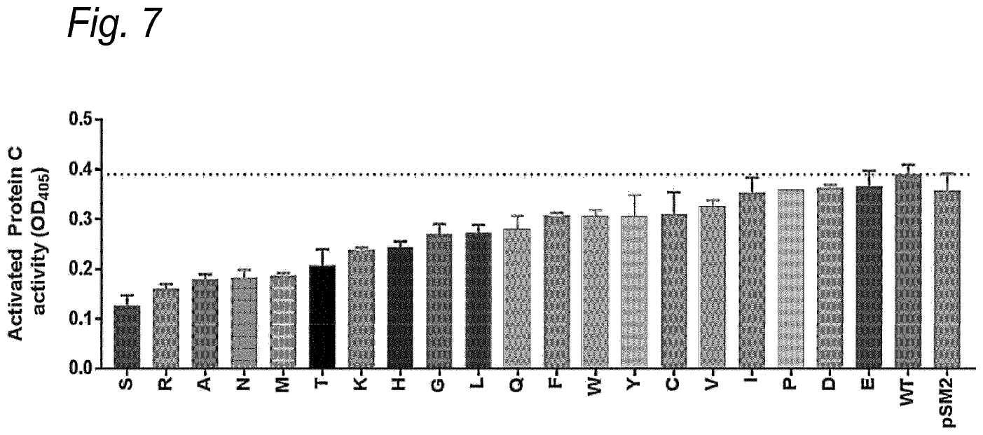

Results are shown in FIG. 3 for the inhibition of Factor XIIa, FIG. 4 for the inhibition of plasma kallikrein, FIG. 5 for the inhibition of plasmin, FIG. 6 for the inhibition of thrombin and FIG. 7 for the inhibition of activated protein C. Results indicate that A, M, R and T are preferred residues at the P1′ position.

What is claimed is:

1. A method of treating a bradykinin-mediated disease, wherein the method comprises administering to a subject in need thereof an effective amount of a modified α1 antitrypsin comprising a reactive center loop (RCL), wherein the residues P4-P1 of the RCL are selected from the group consisting of SMTR (SEQ ID NO: 12), SEAR (SEQ ID NO: 18), SMDR (SEQ ID NO: 23), SLGR (SEQ ID NO: 24), SKGR (SEQ ID NO: 25) and SMHR (SEQ ID NO: 27), wherein the modified α1 antitrypsin demonstrates increased inhibition of plasma kallikrein (PK) as compared to the corresponding unmodified α1 antitrypsin, and wherein the modified α1-antitrypsin more strongly inhibits PK than the modified α1 antitrypsin inhibits either one of thrombin or APC.

2. The method of claim 1, wherein the serpin comprises an amino acid sequence that has at least 70% sequence identity to wild-type serpin α1 AT.

3. The method of claim 1, wherein the mutations increase the inhibition of at least one of an active form of factor XII (FXII) and plasmin as compared to the corresponding unmodified serpin.

4. The method of claim 1, wherein the minimal amount of the serpin that inhibits PK activity by at least 50%, is an amount that inhibits thrombin activity by no more than 15%.

5. The method of claim 4, wherein the minimal amount of the serpin that inhibits PK activity by at least 50%, is an amount that inhibits at least one of:
a) plasmin by at least 15%;
b) an active form of FXII by at least 15%; and,
c) APC by no more than 15%.

6. The method of claim 1, wherein the serpin further has a mutation whereby the P1′ residue of its reactive center loop (RCL) is selected from the group consisting of: Q, F, H, R, K, C, L, Y, N, I, D, W, E, V, M, A, T and P.

7. The method of claim 1, wherein the wild type serpin has been modified to reduce the susceptibility to oxidation.

8. The method of claim 1, wherein the bradykinin-mediated disease is selected from the group consisting of hereditary angioedema, idiopathic angioedema, rheumatoid arthritis, Crohn's disease, ulcerative colitis, lupus, Alzheimer's disease, septic shock, burn injury, brain ischemia/reperfusion injury, cerebral edema, diabetic retinopathy, diabetic nephropathy, macular edema, vasculitis, arterial or venous thrombosis, thrombosis associated with ventricular assist devices or stents, heparin-induced thrombocytopenia with thrombosis, thromboembolic disease, and coronary heart disease with unstable angina pectoris, edema, eye disease, gout, intestinal bowel disease, oral mucositis, neuropathic pain, inflammatory pain, spinal stenosis-degenerative spine disease, post-operative ileus, aortic aneurysm, osteoarthritis, hereditary angioedema, pulmonary embolism, stroke, head trauma or peri-tumor brain edema, sepsis, acute middle cerebral artery (MCA) ischemic event (stroke), restenosis, systemic lupus erythematosis nephritis, an autoimmune disease, an inflammatory disease, a cardiovascular disease, a neurological disease, a disease associated with protein misfolding, a disease associated with angiogenesis, hypertensive nephropathy and diabetic nephropathy, allergic and respiratory diseases, tissue injuries and a poli-trauma.

9. The method of claim 6, wherein the P1′ residue is selected from the group consisting of K, L, Y, I, D, E, A, T, M, R and V.

10. The method of claim 6, wherein the P1′ residue is selected from the group consisting of I and V.

11. The method of claim 8, wherein the restenosis is after angioplasty.

12. The method of claim 8, wherein the inflammatory disease is chronic auto-inflammatory urticaria.

13. The method of claim 8, wherein the respiratory disease is selected from the group consisting of anaphylaxis, asthma, chronic obstructive pulmonary disease, acute respiratory distress syndrome, cystic fibrosis, persistent, rhinitis, urticarial and histamine dependent angioedema.

14. The method of claim 8, wherein the tissue injury is a burn or a chemical injury.

15. The method of claim 8, wherein the poli-trauma is a car crash injury or a battle injury.


SEQUENCE LISTING (360 sequences)

SEQ ID NO: 1 - protein, 394 aa, Homo sapiens
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 2 - protein, 398 aa, Homo sapiens
NSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 3 - protein, 478 aa, Homo sapiens
NPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 4 - protein, 464 aa, Homo sapiens
MEPLGRQLTSGPNQEQVSPLTLLKLGNQEPGGQTALKSPPGVCSRDPTPEQTHRLARAMMAFTADLFSLVAQTSTCPNLILSPLSVALALSHLALGAQNHTLQRLQQVLHAGSGPCLPHLLSRLCQDLGPGAFRLAARMYLQKGFPIKEDFLEQSEQLFGAKPVSLTGKQEDDLANINQWVKEATEGKIQEFLSGLPEDTVLLLLNAIHFQGFWRNKFDPSLTQRDSFHLDEQFTVPVEMMQARTYPLRWFLLEQPEIQVAHFPFKNNMSFVVLVPTHFEWNVSQVLANLSWDTLHPPLVWERPTKVRLPKLYLKHQMDLVATLSQLGLQELFQAPDLRGISEQSLVVSGVQHQSTLELSEVGVEAAAATSIAMSRMSLSSFSVNRPFLFFIFEDTTGLPLFVGSVRNPNPSAPRELKEQQDSPGNKDFLQSLKGFPRGDKLFGPDLKLVPPMEEDYPQFGSPK

SEQ ID NO: 5 - protein, 432 aa, Homo sapiens [sequence omitted]

SEQ ID NO: 6 - protein, 480 aa, Homo sapiens
GSKGPLDQLEKGGETAQSADPQWEQLNNKNLSMPLLPADFHKENTVTNDWIPEGEEDDDYLDLEKIFSEDDDYIDIVDSLSVSPTDSDVSAGNILQLFHGKSRIQRLNILNAKFAFNLYRVLKDQVNTFDNIFIAPVGISTAMGMISLGLKGETHEQVHSILHFKDFVNASSKYEITTIHNLFRKLTHRLFRRNFGYTLRSVNDLYIQKQFPILLDFKTKVREYYFAEAQIADFSDPAFISKTNNHIMKLTKGLIKDALENIDPATQMMILNCIYFKGSWVNKFPVEMTHNHNFRLNEREVVKVSMMQTKGNFLAANDQELDCDILQLEYVGGISMLIVVPHKMSGMKTLEAQLTPRVVERWQKSMTNRTREVLLPKFKLEKNYNLVESLKLMGIRMLFDKNGNMAGISDQRIAIDLFKHQGTITVNEEGTQATTVTTVGFMPLSTQVRFTVDRPFLFLIYEHRTSCLLFMGRVANPSRS

SEQ ID NO: 7 - protein, 387 aa, Homo sapiens
HRHHPREMKKRVEDLHVGATVAPSSRRDFTFDLYRALASAAPSQSIFFSPVSISMSLAMLSLGAGSSTKMQILEGLGLNLQKSSEKELHRGFQQLLQELNQPRDGFQLSLGNALFTDLVVDLQDTFVSAMKTLYLADTFPTNFRDSAGAMKQINDYVAKQTKGKIVDLLKNLDSNAVVIMVNYIFFKAKWETSFNHKGTQEQDFYVTSETVVRVPMMSREDQYHYLLDRNLSCRVVGVPYQGNATALFILPSEGKMQQVENGLSEKTLRKWLKMFKKRQLELYLPKFSIEGSYQLEKVLPSLGISNVFTSHADLSGISNHSNIQVSEMVHKAVVEVDESGTRAAAATGTIFTFRSARLNSQRLVFNRPFLMFIVDNNILFLGKVNRP

SEQ ID NO: 8 - protein, 407 aa, Homo sapiens
QLHVEHDGESCSNSSHQQILETGEGSPSLKIAPANADFAFRFYYLIASETPGKNIFFSPLSISAAYAMLSLGACSHSRSQILEGLGFNLTELSESDVHRGFQHLLHTLNLPGHGLETRVGSALFLSHNLKFLAKFLNDTMAVYEAKLFHTNFYDTVGTIQLINDHVKKETRGKIVDLVSELKKDVLMVLVNYIYFKALWEKPFISSRTTPKDFYVDENTTVRVPMMLQDQEHHWYLHDRYLPCSVLRMDYKGDATVFFILPNQGKMREIEEVLTPEMLMRWNNLLRKRNFYKKLELHLPKFSISGSYVLDQILPRLGFTDLFSKWADLSGITKQQKLEASKSFHKATLDVDEAGTEAAAATSFAIKFFSAQTNRHILRFNRPFLVVIFSTSTQSVLFLGKVVDPTKP

SEQ ID NO: 9 - protein, 379 aa, Homo sapiens [sequence omitted]

SEQ ID NO: 10 - protein, 379 aa, Homo sapiens
SHFNPLSLEELGSNTGIQVFNQIVKSRPHDNIVISPHGIASVLGMLQLGADGRTKKQLAMVMRYGVNGVGKILKKINKAIVSKKNKDIVTVANAVFVKNASEIEVPFVTRNKDVFQCEVRNVNFEDPASACDSINAWVKNETRDMIDNLLSPDLIDGVLTRLVLVNAVYFKGLWKSRFQPENTKKRTFVAADGKSYQVPMLAQLSVFRCGSTSAPNDLWYNFIELPYHGESISMLIALPTESSTPLSAIIPHISTKTIDSWMSIMVPKRVQVILPKFTAVAQTDLKEPLKVLGITDMFDSSKANFAKITTGSENLHVSHILQKAKIEVSEDGTKASAATTAILIARSSPPWFIVDRPFLFFIRHNPTGAVLFMGQINKP

SEQ ID NO: 11 - protein, 423 aa, Homo sapiens
LAPSPQSPETPAPQNQTSRVVQAPKEEEEDEQEASEEKASEEEKAWLMASRQQLAKETSNFGFSLLRKISMRHDGNMVFSPFGMSLAMTGLMLGATGPTETQIKRGLHLQALKPTKPGLLPSLFKGLRETLSRNLELGLTQGSFAFIHKDFDVKETFFNLSKRYFDTECVPMNFRNASQAKRLMNHYINKETRGKIPKLFDEINPETKLILVDYILFKGKWLTPFDPVFTEVDTFHLDKYKTIKVPMMYGAGKFASTFDKNFRCHVLKLPYQGNATMLVVLMEKMGDHLALEDYLTTDLVETWLRNMKTRNMEVFFPKFKLDQKYEMHELLRQMGIRRIFSPFADLSELSATGRNLQVSRVLQRTVIEVDERGTEAVAGILSEITAYSMPPVIKVDRPFHFMIYEETSGMLLFLGRVVNPTLL

SEQ ID NO: 12 - protein, 4 aa, synthetic construct
  REGION 1..4: note = peptide
SMTR

SEQ ID NO: 13 - protein, 4 aa, synthetic construct
  REGION 1..4: note = peptide
SGQR

SEQ ID NO: 14 - protein, 4 aa, synthetic construct
  REGION 1..4: note = peptide
SVTR

SEQ ID NO: 15 - protein, 4 aa, synthetic construct
  REGION 1..4: note = peptide
SATR

SEQ ID NO: 16 - protein, 4 aa, synthetic construct
  REGION 1..4: note = peptide
SFNR

SEQ ID NO: 17 - protein, 4 aa, synthetic construct
  REGION 1..4: note = Peptide
SWKK

SEQ ID NO: 18 - protein, 4 aa, synthetic construct
  REGION 1..4: note = peptide
SEAR

SEQ ID NO: 19 - protein, 4 aa, synthetic construct
  REGION 1..4: note = peptide
SVVK

SEQ ID NO: 20 - protein, 4 aa, synthetic construct
  REGION 1..4: note = Peptide
SDYK

SEQ ID NO: 21 - protein, 4 aa, synthetic construct
  REGION 1..4: note = peptide
SVRK

SEQ ID NO: 22 - protein, 4 aa, synthetic construct
  REGION 1..4: note = peptide
SPRR

SEQ ID NO: 23 - protein, 4 aa, synthetic construct
  REGION 1..4: note = peptide
SMDR

SEQ ID NO: 24 - protein, 4 aa, synthetic construct
  REGION 1..4: note = peptide
SLGR

SEQ ID NO: 25 - protein, 4 aa, synthetic construct
  REGION 1..4: note = peptide
SKGR

SEQ ID NO: 26 - protein, 4 aa, synthetic construct
  REGION 1..4: note = Peptide
SGNR

SEQ ID NO: 27 - protein, 4 aa, synthetic construct
  REGION 1..4: note = Peptide
SMHR

SEQ ID NO: 28 - protein, 4 aa, synthetic construct
  REGION 1..4: note = peptide
SLLR

SEQ ID NO: 29 - protein, 5 aa, synthetic construct
  REGION 1..5: note = Peptide
SMTRQ

SEQ ID NO: 30 - protein, 5 aa, synthetic construct
  REGION 1..5: note = peptide
SMTRF

SEQ ID NO: 31 - protein, 5 aa, synthetic construct
  REGION 1..5: note = peptide
SMTRH

SEQ ID NO: 32 - protein, 5 aa, synthetic construct
  REGION 1..5: note = Peptide
SMTRR

SEQ ID NO: 33 - protein, 5 aa, synthetic construct
  REGION 1..5: note = Peptide
SMTRK

SEQ ID NO: 34 - protein, 5 aa, synthetic construct
  REGION 1..5: note = Peptide
SMTRC

SEQ ID NO: 35 - protein, 5 aa, synthetic construct
  REGION 1..5: note = Peptide
SMTRL

SEQ ID NO: 36 - protein, 5 aa, synthetic construct
  REGION 1..5: note = Peptide
SMTRY

SEQ ID NO: 37 - protein, 5 aa, synthetic construct
  REGION 1..5: note = peptide
SMTRN

SEQ ID NO: 38 - protein, 5 aa, synthetic construct
  REGION 1..5: note = peptide
SMTRI

SEQ ID NO: 39 - protein, 5 aa, synthetic construct
  REGION 1..5: note = Peptide
SMTRD

SEQ ID NO: 40 - protein, 5 aa, synthetic construct
  REGION 1..5: note = peptide
SMTRW

SEQ ID NO: 41 - protein, 5 aa, synthetic construct
  REGION 1..5: note = peptide
SMTRE

SEQ ID NO: 42 - protein, 5 aa, synthetic construct
  REGION 1..5: note = peptide
SMTRV

SEQ ID NO: 43 - protein, 5 aa, synthetic construct
  REGION 1..5: note = peptide
SMTRM

SEQ ID NO: 44 - protein, 5 aa, synthetic construct
  REGION 1..5: note = peptide
SMTRA

SEQ ID NO: 45 - protein, 5 aa, synthetic construct
  REGION 1..5: note = peptide
SMTRT

SEQ ID NO: 46 - protein, 5 aa, synthetic construct
  REGION 1..5: note = peptide
SMTRP

SEQ ID NO: 47 - protein, 5 aa, synthetic construct
  REGION 1..5: note = peptide
SGQRQ

SEQ ID NO: 48 - protein, 5 aa, synthetic construct
  REGION 1..5: note = peptide
SGQRF

SEQ ID NO: 49 - protein, 5 aa, synthetic construct
  REGION 1..5: note = peptide
SGQRH

SEQ ID NO: 50 - protein, 5 aa, synthetic construct
  REGION 1..5: note = peptide
SGQRR

SEQ ID NO: 51 - protein, 5 aa, synthetic construct
  REGION 1..5: note = peptide
SGQRK

SEQ ID NO: 52 - protein, 5 aa, synthetic construct
  REGION 1..5: note = peptide
SGQRC

SEQ ID NO: 53 - protein, 5 aa, synthetic construct
  REGION 1..5: note = peptide
SGQRL

SEQ ID NO: 54 - protein, 5 aa, synthetic construct
  REGION 1..5: note = peptide
SGQRY

SEQ ID NO: 55 - protein, 5 aa, synthetic construct
  REGION 1..5: note = peptide
SGQRN

SEQ ID NO: 56 - protein, 5 aa, synthetic construct
  REGION 1..5: note = peptide
SGQRI

SEQ ID NO: 57 - protein, 5 aa, synthetic construct
  REGION 1..5: note = peptide
SGQRD

SEQ ID NO: 58 - protein, 5 aa, synthetic construct
  REGION 1..5: note = peptide
SGQRW

SEQ ID NO: 59 - protein, 5 aa, synthetic construct
  REGION 1..5: note = peptide
SGQRE

SEQ ID NO: 60 - protein, 5 aa, synthetic construct
  REGION 1..5: note = peptide
SGQRV

SEQ ID NO: 61 - protein, 5 aa, synthetic construct
  REGION 1..5: note = peptide
SGQRM

SEQ ID NO: 62 - protein, 5 aa, synthetic construct
  REGION 1..5: note = peptide
SGQRA

SEQ ID NO: 63 - protein, 5 aa, synthetic construct
  REGION 1..5: note = peptide
SGQRT

SEQ ID NO: 64 - protein, 5 aa, synthetic construct
  REGION 1..5: note = peptide
SGQRP

SEQ ID NO: 65 - protein, 5 aa, synthetic construct
  REGION 1..5: note = peptide
SVTRQ

SEQ ID NO: 66 - protein, 5 aa, synthetic construct
  REGION 1..5: note = peptide
SVTRF

SEQ ID NO: 67 - protein, 5 aa, synthetic construct
  REGION 1..5: note = peptide
SVTRH

SEQ ID NO: 68 - protein, 5 aa, synthetic construct
  REGION 1..5: note = peptide
SVTRR

SEQ ID NO: 69 - protein, 5 aa, synthetic construct
  REGION 1..5: note = peptide
SVTRK

SEQ ID NO: 70 - protein, 5 aa, synthetic construct
  REGION 1..5: note = peptide
SVTRC

SEQ ID NO: 71 - protein, 5 aa, synthetic construct
  REGION 1..5: note = peptide
SVTRL

SEQ ID NO: 72 - protein, 5 aa, synthetic construct
  REGION 1..5: note = peptide
SVTRY

SEQ ID NO: 73 - protein, 5 aa, synthetic construct
  REGION 1..5: note = peptide
SVTRN

SEQ ID NO: 74 - protein, 5 aa, synthetic construct
  REGION 1..5: note = peptide
SVTRI

SEQ ID NO: 75 - protein, 5 aa, synthetic construct
  REGION 1..5: note = peptide
SVTRD

SEQ ID NO: 76 - protein, 5 aa, synthetic construct
  REGION 1..5: note = peptide
SVTRW

SEQ ID NO: 77 - protein, 5 aa, synthetic construct
  REGION 1..5: note = peptide
SVTRE

SEQ ID NO: 78 - protein, 5 aa, synthetic construct
  REGION 1..5: note = peptide
SVTRV

SEQ ID NO: 79 - protein, 5 aa, synthetic construct
  REGION 1..5: note = peptide
SVTRM

SEQ ID NO: 80 - protein, 5 aa, synthetic construct
  REGION 1..5: note = peptide
SVTRA

SEQ ID NO: 81 - protein, 5 aa, synthetic construct
  REGION 1..5: note = peptide
SVTRT

SEQ ID NO: 82 - protein, 5 aa, synthetic construct
  REGION 1..5: note = peptide
SVTRP

SEQ ID NO: 83 - protein, 5 aa, synthetic construct
  REGION 1..5: note = peptide
SATRQ

SEQ ID NO: 84 - protein, 5 aa, synthetic construct
  REGION 1..5: note = peptide
SATRF

SEQ ID NO: 85 - protein, 5 aa, synthetic construct
  REGION 1..5: note = peptide
SATRH

SEQ ID NO: 86 - protein, 5 aa, synthetic construct
  REGION 1..5: note = peptide
SATRR

SEQ ID NO: 87 - protein, 5 aa, synthetic construct
  REGION 1..5: note = peptide
SATRK

SEQ ID NO: 88 - protein, 5 aa, synthetic construct
  REGION 1..5: note = peptide
SATRC

SEQ ID NO: 89 - protein, 5 aa, synthetic construct
  REGION 1..5: note = peptide
SATRL

SEQ ID NO: 90 - protein, 5 aa, synthetic construct
  REGION 1..5: note = peptide
SATRY

SEQ ID NO: 91 - protein, 5 aa, synthetic construct
  REGION 1..5: note = peptide
SATRN

SEQ ID NO: 92 - protein, 5 aa, synthetic construct
  REGION 1..5: note = peptide
SATRI

SEQ ID NO: 93 - protein, 5 aa, synthetic construct
  REGION 1..5: note = peptide
SATRD

SEQ ID NO: 94 - protein, 5 aa, synthetic construct
  REGION 1..5: note = peptide
SATRW

SEQ ID NO: 95 - protein, 5 aa, synthetic construct
  REGION 1..5: note = peptide
SATRE

SEQ ID NO: 96 - protein, 5 aa, synthetic construct
  REGION 1..5: note = peptide
SATRV

SEQ ID NO: 97 - protein, 5 aa, synthetic construct
  REGION 1..5: note = SATRM
SATRM

SEQ ID NO: 98 - protein, 5 aa, synthetic construct
  REGION 1..5: note = peptide
SATRA

SEQ ID NO: 99 - protein, 5 aa, synthetic construct
  REGION 1..5: note = peptide
SATRT

SEQ ID NO: 100 - protein, 5 aa, synthetic construct
  REGION 1..5: note = peptide
SATRP

SEQ ID NO: 101 - protein, 5 aa, synthetic construct
  REGION 1..5: note = peptide
SFNRQ

SEQ ID NO: 102 - protein, 5 aa, synthetic construct
  REGION 1..5: note = peptide
SFNRF

SEQ ID NO: 103 - protein, 5 aa, synthetic construct
  REGION 1..5: note = peptide
SFNRH

SEQ ID NO: 104 - protein, 5 aa, synthetic construct
  REGION 1..5: note = peptide
SFNRR

SEQ ID NO: 105 - protein, 5 aa, synthetic construct
  REGION 1..5: note = peptide
SFNRK

SEQ ID NO: 106 - protein, 5 aa, synthetic construct
  REGION 1..5: note = peptide
SFNRC

SEQ ID NO: 107 - protein, 5 aa, synthetic construct
  REGION 1..5: note = peptide
SFNRL

SEQ ID NO: 108 - protein, 5 aa, synthetic construct
  REGION 1..5: note = peptide
SFNRY

SEQ ID NO: 109 - protein, 5 aa, synthetic construct
  REGION 1..5: note = artificial
SFNRN

SEQ ID NO: 110 - protein, 5 aa, synthetic construct
  REGION 1..5: note = peptide
SFNRI

SEQ ID NO: 111 - protein, 5 aa, synthetic construct
  REGION 1..5: note = peptide
SFNRD

SEQ ID NO: 112 - protein, 5 aa, synthetic construct
  REGION 1..5: note = peptide
SFNRW

SEQ ID NO: 113 - protein, 5 aa, synthetic construct
  REGION 1..5: note = peptide
SFNRE

SEQ ID NO: 114 - protein, 5 aa, synthetic construct
  REGION 1..5: note = peptide
SFNRV

SEQ ID NO: 115 - protein, 5 aa, synthetic construct
  REGION 1..5: note = peptide
SFNRM

SEQ ID NO: 116 - protein, 5 aa, synthetic construct
  REGION 1..5: note = peptide
SFNRA

SEQ ID NO: 117 - protein, 5 aa, synthetic construct
  REGION 1..5: note = peptide
SFNRT

SEQ ID NO: 118 - protein, 5 aa, synthetic construct
  REGION 1..5: note = peptide
SFNRP

SEQ ID NO: 119 - protein, 5 aa, synthetic construct
  REGION 1..5: note = peptide
SWKKQ

SEQ ID NO: 120 - protein, 5 aa, synthetic construct
  REGION 1..5: note = peptide
SWKKF

SEQ ID NO: 121 - protein, 5 aa, synthetic construct
  REGION 1..5: note = peptide
SWKKH

SEQ ID NO: 122 - protein, 5 aa, synthetic construct
  REGION 1..5: note = peptide
SWKKR

SEQ ID NO: 123 - protein, 5 aa, synthetic construct
  REGION 1..5: note = peptide
SWKKK

SEQ ID NO: 124 - protein, 5 aa, synthetic construct
  REGION 1..5: note = peptide
SWKKC

SEQ ID NO: 125 - protein, 5 aa, synthetic construct
  REGION 1..5: note = peptide
SWKKL

SEQ ID NO: 126 - protein, 5 aa, synthetic construct
  REGION 1..5: note = peptide
SWKKY

SEQ ID NO: 127 - protein, 5 aa, synthetic construct
  REGION 1..5: note = peptide
SWKKN

SEQ ID NO: 128 - protein, 5 aa, synthetic construct
  REGION 1..5: note = peptide
SWKKI

SEQ ID NO: 129 - protein, 5 aa, synthetic construct
  REGION 1..5: note = peptide
SWKKD

SEQ ID NO: 130 - protein, 5 aa, synthetic construct
  REGION 1..5: note = peptide
SWKKW

SEQ ID NO: 131 - protein, 5 aa, synthetic construct
  REGION 1..5: note = peptide
SWKKE

SEQ ID NO: 132 - protein, 5 aa, synthetic construct
  REGION 1..5: note = peptide
SWKKV

SEQ ID NO: 133 - protein, 5 aa, synthetic construct
  REGION 1..5: note = SWKKM
SWKKM

SEQ ID NO: 134 - protein, 5 aa, synthetic construct
  REGION 1..5: note = peptide
SWKKA

SEQ ID NO: 135 - protein, 5 aa, synthetic construct
  REGION 1..5: note = peptide
SWKKT

SEQ ID NO: 136 - protein, 5 aa, synthetic construct
  REGION 1..5: note = peptide
SWKKP

SEQ ID NO: 137 - protein, 5 aa, synthetic construct
  REGION 1..5: note = peptide
SEARQ

SEQ ID NO: 138 - protein, 5 aa, synthetic construct
  REGION 1..5: note = peptide
SEARF

SEQ ID NO: 139 - protein, 5 aa, synthetic construct
  REGION 1..5: note = peptide
SEARH

SEQ ID NO: 140 - protein, 5 aa, synthetic construct
  REGION 1..5: note = peptide
SEARR

SEQ ID NO: 141 - protein, 5 aa, synthetic construct
  REGION 1..5: note = peptide
SEARK

SEQ ID NO: 142 - protein, 5 aa, synthetic construct
  REGION 1..5: note = peptide
SEARC

SEQ ID NO: 143 - protein, 5 aa, synthetic construct
  REGION 1..5: note = peptide
SEARL

SEQ ID NO: 144 - protein, 5 aa, synthetic construct
  REGION 1..5: note = peptide
SEARY

SEQ ID NO: 145 - protein, 5 aa, synthetic construct
  REGION 1..5: note = peptide
SEARN

SEQ ID NO: 146 - protein, 5 aa, synthetic construct
  REGION 1..5: note = peptide
SEARI

SEQ ID NO: 147 - protein, 5 aa, synthetic construct
  REGION 1..5: note = peptide
SEARD

SEQ ID NO: 148 - protein, 5 aa, synthetic construct
  REGION 1..5: note = peptide
SEARW

SEQ ID NO: 149 - protein, 5 aa, synthetic construct
  REGION 1..5: note = peptide
SEARE

SEQ ID NO: 150 - protein, 5 aa, synthetic construct
  REGION 1..5: note = peptide
SEARV

SEQ ID NO: 151 - protein, 5 aa, synthetic construct
  REGION 1..5: note = peptide
SEARM

SEQ ID NO: 152 - protein, 5 aa, synthetic construct
  REGION 1..5: note = peptide
SEARA

SEQ ID NO: 153 - protein, 5 aa, synthetic construct
  REGION 1..5: note = peptide
SEART

SEQ ID NO: 154 - protein, 5 aa, synthetic construct
  REGION 1..5: note = peptide
SEARP

SEQ ID NO: 155 - protein, 5 aa, synthetic construct
  REGION 1..5: note = peptide
SVVKQ

SEQ ID NO: 156 - protein, 5 aa, synthetic construct
  REGION 1..5: note = peptide
SVVKF

SEQ ID NO: 157 - protein, 5 aa, synthetic construct
  REGION 1..5: note = peptide
SVVKH

SEQ ID NO: 158 - protein, 5 aa, synthetic construct
  REGION 1..5: note = peptide
SVVKR

SEQ ID NO: 159 - protein, 5 aa, synthetic construct
  REGION 1..5: note = petide
SVVKK

SEQ ID NO: 160 - protein, 5 aa, synthetic construct
  REGION 1..5: note = peptide
SVVKC

SEQ ID NO: 161 - protein, 5 aa, synthetic construct
  REGION 1..5: note = peptide
SVVKL

SEQ ID NO: 162 - protein, 5 aa, synthetic construct
  REGION 1..5: note = peptide
SVVKY

SEQ ID NO: 163 - protein, 5 aa, synthetic construct
  REGION 1..5: note = peptide
SVVKN

SEQ ID NO: 164 - protein, 5 aa, synthetic construct
  REGION 1..5: note = peptide
SVVKI

SEQ ID NO: 165 - protein, 5 aa, synthetic construct
  REGION 1..5: note = peptide
SVVKD

SEQ ID NO: 166 - protein, 5 aa, synthetic construct
  REGION 1..5: note = peptide
SVVKW

SEQ ID NO: 167 - protein, 5 aa, synthetic construct
  REGION 1..5: note = peptide
SVVKE

SEQ ID NO: 168 - protein, 5 aa, synthetic construct
  REGION 1..5: note = peptide
SVVKV

SEQ ID NO: 169 - protein, 5 aa, synthetic construct
  REGION 1..5: note = peptide
SVVKM

SEQ ID NO: 170 - protein, 5 aa, synthetic construct
  REGION 1..5: note = peptide
SVVKA

SEQ ID NO: 171 - protein, 5 aa, synthetic construct
  REGION 1..5: note = peptide
SVVKT

SEQ ID NO: 172 - protein, 5 aa, synthetic construct
  REGION 1..5: note = peptide
SVVKP

SEQ ID NO: 173 - protein, 5 aa, synthetic construct
  REGION 1..5: note = peptide
SDYKQ

SEQ ID NO: 174 - protein, 5 aa, synthetic construct
  REGION 1..5: note = peptide
SDYKF

SEQ ID NO: 175 - protein, 5 aa, synthetic construct
  REGION 1..5: note = peptide
SDYKH

SEQ ID NO: 176 - protein, 5 aa, synthetic construct
  REGION 1..5: note = peptide
SDYKR

SEQ ID NO: 177 - protein, 5 aa, synthetic construct
  REGION 1..5: note = peptide
SDYKK

SEQ ID NO: 178 - protein, 5 aa, synthetic construct
  REGION 1..5: note = peptide
SDYKC

SEQ ID NO: 179 - protein, 5 aa, synthetic construct
  REGION 1..5: note = peptide
SDYKL

SEQ ID NO: 180 - protein, 5 aa, synthetic construct
  REGION 1..5: note = peptide
SDYKY

SEQ ID NO: 181 - protein, 5 aa, synthetic construct
  REGION 1..5: note = peptide
SDYKN

SEQ ID NO: 182 - protein, 5 aa, synthetic construct
  REGION 1..5: note = peptide
SDYKI

SEQ ID NO: 183 - protein, 5 aa, synthetic construct
  REGION 1..5: note = peptide
SDYKD

SEQ ID NO: 184 - protein, 5 aa, synthetic construct
  REGION 1..5: note = peptide
SDYKW

SEQ ID NO: 185 - protein, 5 aa, synthetic construct
  REGION 1..5: note = peptide
SDYKE

SEQ ID NO: 186 - protein, 5 aa, synthetic construct
  REGION 1..5: note = peptide
SDYKV

SEQ ID NO: 187 - protein, 5 aa, synthetic construct
  REGION 1..5: note = peptide
SDYKM

SEQ ID NO: 188 - protein, 5 aa, synthetic construct
  REGION 1..5: note = peptide
SDYKA

SEQ ID NO: 189 - protein, 5 aa, synthetic construct
  REGION 1..5: note = peptide
SDYKT

SEQ ID NO: 190 - protein, 5 aa, synthetic construct
  REGION 1..5: note = peptide
SDYKP

SEQ ID NO: 191 - protein, 5 aa, synthetic construct
  REGION 1..5: note = peptide
SVRKQ

SEQ ID NO: 192 - protein, 5 aa, synthetic construct
  REGION 1..5: note = peptide
SVRKF

SEQ ID NO: 193 - protein, 5 aa, synthetic construct
  REGION 1..5: note = peptide
SVRKH

SEQ ID NO: 194 - protein, 5 aa, synthetic construct
  REGION 1..5: note = peptide
SVRKR

SEQ ID NO: 195 - protein, 5 aa, synthetic construct
  REGION 1..5: note = peptide
SVRKK

SEQ ID NO: 196 - protein, 5 aa, synthetic construct
  REGION 1..5: note = peptide
SVRKC

SEQ ID NO: 197 - protein, 5 aa, synthetic construct
  REGION 1..5: note = peptide
SVRKL

SEQ ID NO: 198 - protein, 5 aa, synthetic construct
  REGION 1..5: note = peptide
SVRKY

SEQ ID NO: 199 - protein, 5 aa, synthetic construct
  REGION 1..5: note = peptide
SVRKN

SEQ ID NO: 200 - protein, 5 aa, synthetic construct
  REGION 1..5: note = peptide
SVRKI

SEQ ID NO: 201 - protein, 5 aa, synthetic construct
  REGION 1..5: note = peptide
SVRKD

SEQ ID NO: 202 - protein, 5 aa, synthetic construct
  REGION 1..5: note = peptide
SVRKW

SEQ ID NO: 203 - protein, 5 aa, synthetic construct
  REGION 1..5: note = peptide
SVRKE

SEQ ID NO: 204 - protein, 5 aa, synthetic construct
  REGION 1..5: note = peptide
SVRKV

SEQ ID NO: 205 - protein, 5 aa, synthetic construct
  REGION 1..5: note = peptide
SVRKM

SEQ ID NO: 206 - protein, 5 aa, synthetic construct
  REGION 1..5: note = peptide
SVRKA

SEQ ID NO: 207 - protein, 5 aa, synthetic construct
  REGION 1..5: note = peptide
SVRKT

SEQ ID NO: 208 - protein, 5 aa, synthetic construct
  REGION 1..5: note = peptide
SVRKP

SEQ ID NO: 209 - protein, 5 aa, synthetic construct
  REGION 1..5: note = peptide
SPRRQ

SEQ ID NO: 210 - protein, 5 aa, synthetic construct
  REGION 1..5: note = peptide
SPRRF

SEQ ID NO: 211 - protein, 5 aa, synthetic construct
  REGION 1..5: note = peptide
SPRRH

SEQ ID NO: 212 - protein, 5 aa, synthetic construct
  REGION 1..5: note = peptide
SPRRR

SEQ ID NO: 213 - protein, 5 aa, synthetic construct
  REGION 1..5: note = peptide
SPRRK

SEQ ID NO: 214 - protein, 5 aa, synthetic construct
  REGION 1..5: note = peptide
SPRRC

SEQ ID NO: 215 - protein, 5 aa, synthetic construct
  REGION 1..5: note = peptide
SPRRL

SEQ ID NO: 216 - protein, 5 aa, synthetic construct
  REGION 1..5: note = peptide
SPRRY

SEQ ID NO: 217 - protein, 5 aa, synthetic construct
  REGION 1..5: note = peptide
SPRRN

SEQ ID NO: 218 - protein, 5 aa, synthetic construct
  REGION 1..5: note = peptide
SPRRI

SEQ ID NO: 219 - protein, 5 aa, synthetic construct
  REGION 1..5: note = peptide
SPRRD

SEQ ID NO: 220 - protein, 5 aa, synthetic construct
  REGION 1..5: note = peptide
SPRRW

SEQ ID NO: 221 - protein, 5 aa, synthetic construct
  REGION 1..5: note = peptide
SPRRE

SEQ ID NO: 222 - protein, 5 aa, synthetic construct
  REGION 1..5: note = peptide
SPRRV

SEQ ID NO: 223 - protein, 5 aa, synthetic construct
  REGION 1..5: note = peptide
SPRRM

SEQ ID NO: 224 - protein, 5 aa, synthetic construct
  REGION 1..5: note = peptide
SPRRA

SEQ ID NO: 225 - protein, 5 aa, synthetic construct
  REGION 1..5: note = peptide
SPRRT

SEQ ID NO: 226 - protein, 5 aa, synthetic construct
  REGION 1..5: note = peptide
SPRRP

SEQ ID NO: 227 - protein, 5 aa, synthetic construct
  REGION 1..5: note = peptide
SMDRQ

SEQ ID NO: 228 - protein, 5 aa, synthetic construct
  REGION 1..5: note = peptide
SMDRF

SEQ ID NO: 229 - protein, 5 aa, synthetic construct
  REGION 1..5: note = peptide
SMDRH

SEQ ID NO: 230 - protein, 5 aa, synthetic construct
  REGION 1..5: note = peptide
SMDRR

SEQ ID NO: 231 - protein, 5 aa, synthetic construct
  REGION 1..5: note = peptide
SMDRK

SEQ ID NO: 232 - protein, 5 aa, synthetic construct
  REGION 1..5: note = peptide
SMDRC

SEQ ID NO: 233 - protein, 5 aa, synthetic construct
  REGION 1..5: note = peptide
SMDRL

SEQ ID NO: 234 - protein, 5 aa, synthetic construct
  REGION 1..5: note = peptide
SMDRY

SEQ ID NO: 235 - protein, 5 aa, synthetic construct
  REGION 1..5: note = peptide
SMDRN

SEQ ID NO: 236 - protein, 5 aa, synthetic construct
  REGION 1..5: note = peptide
SMDRI

SEQ ID NO: 237 - protein, 5 aa, synthetic construct
  REGION 1..5: note = peptide
SMDRD

SEQ ID NO: 238 - protein, 5 aa, synthetic construct
  REGION 1..5: note = peptide
SMDRW

SEQ ID NO: 239 - protein, 5 aa, synthetic construct
  REGION 1..5: note = peptide
SMDRE

SEQ ID NO: 240 - protein, 5 aa, synthetic construct
  REGION 1..5: note = peptide
SMDRV

SEQ ID NO: 241 - protein, 5 aa, synthetic construct
  REGION 1..5: note = peptide
SMDRM

SEQ ID NO: 242 - protein, 5 aa, synthetic construct
  REGION 1..5: note = peptide
SMDRA

SEQ ID NO: 243 - protein, 5 aa, synthetic construct
  REGION 1..5: note = peptide
SMDRT

SEQ ID NO: 244 - protein, 5 aa, synthetic construct
  REGION 1..5: note = peptide
SMDRP

SEQ ID NO: 245 - protein, 5 aa, synthetic construct
  REGION 1..5: note = peptide
SLGRQ

SEQ ID NO: 246 - protein, 5 aa, synthetic construct
  REGION 1..5: note = peptide
SLGRF

SEQ ID NO: 247 - protein, 5 aa, synthetic construct
  REGION 1..5: note = peptide
SLGRH

SEQ ID NO: 248 - protein, 5 aa, synthetic construct
  REGION 1..5: note = peptide
SLGRR

SEQ ID NO: 249 - protein, 5 aa, synthetic construct
  REGION 1..5: note = peptide
SLGRK

SEQ ID NO: 250 - protein, 5 aa, synthetic construct
  REGION 1..5: note = peptide
SLGRC

SEQ ID NO: 251 - protein, 5 aa, synthetic construct
  REGION 1..5: note = peptide
SLGRL

SEQ ID NO: 252 - protein, 5 aa, synthetic construct
  REGION 1..5: note = peptide
SLGRY

SEQ ID NO: 253 - protein, 5 aa, synthetic construct
  REGION 1..5: note = peptide
SLGRN

SEQ ID NO: 254 - protein, 5 aa, synthetic construct
  REGION 1..5: note = peptide
SLGRI

SEQ ID NO: 255 - protein, 5 aa, synthetic construct
  REGION 1..5: note = peptide
SLGRD

SEQ ID NO: 256 - protein, 5 aa, synthetic construct
  REGION 1..5: note = peptide
SLGRW

SEQ ID NO: 257 - protein, 5 aa, synthetic construct
  REGION 1..5: note = peptide
SLGRE

SEQ ID NO: 258 - protein, 5 aa, synthetic construct
  REGION 1..5: note = peptide
SLGRV

SEQ ID NO: 259 - protein, 5 aa, synthetic construct
  REGION 1..5: note = peptide
SLGRM

SEQ ID NO: 260 - protein, 5 aa, synthetic construct
  REGION 1..5: note = peptide
SLGRA

SEQ ID NO: 261 - protein, 5 aa, synthetic construct
  REGION 1..5: note = peptide
SLGRT

SEQ ID NO: 262 - protein, 5 aa, synthetic construct
  REGION 1..5: note = peptide
SLGRP

SEQ ID NO: 263 - protein, 5 aa, synthetic construct
  REGION 1..5: note = peptide
SKGRQ

SEQ ID NO: 264 - protein, 5 aa, synthetic construct
  REGION 1..5: note = peptide
SKGRF

SEQ ID NO: 265 - protein, 5 aa, synthetic construct
  REGION 1..5: note = peptide
SKGRH

SEQ ID NO: 266 - protein, 5 aa, synthetic construct
  REGION 1..5: note = peptide
SKGRR

SEQ ID NO: 267 - protein, 5 aa, synthetic construct
  REGION 1..5: note = peptide
SKGRK

SEQ ID NO: 268 - protein, 5 aa, synthetic construct
  REGION 1..5: note = peptide
SKGRC

SEQ ID NO: 269 - protein, 5 aa, synthetic construct
  REGION 1..5: note = peptide
SKGRL

SEQ ID NO: 270 - protein, 5 aa, synthetic construct
  REGION 1..5: note = peptide
SKGRY

SEQ ID NO: 271 - protein, 5 aa, synthetic construct
  REGION 1..5: note = peptide
SKGRN

SEQ ID NO: 272 - protein, 5 aa, synthetic construct
  REGION 1..5: note = peptide
SKGRI

SEQ ID NO: 273 - protein, 5 aa, synthetic construct
  REGION 1..5: note = peptide
SKGRD

SEQ ID NO: 274 - protein, 5 aa, synthetic construct
  REGION 1..5: note = peptide
SKGRW

SEQ ID NO: 275 - protein, 5 aa, synthetic construct
  REGION 1..5: note = peptide
SKGRE

SEQ ID NO: 276 - protein, 5 aa, synthetic construct
  REGION 1..5: note = peptide
SKGRV

SEQ ID NO: 277 - protein, 5 aa, synthetic construct
  REGION 1..5: note = peptide
SKGRM

SEQ ID NO: 278 - protein, 5 aa, synthetic construct
  REGION 1..5: note = peptide
SKGRA

SEQ ID NO: 279 - protein, 5 aa, synthetic construct
  REGION 1..5: note = peptide
SKGRT

SEQ ID NO: 280 - protein, 5 aa, synthetic construct
  REGION 1..5: note = peptide
SKGRP

SEQ ID NO: 281 - protein, 5 aa, synthetic construct
  REGION 1..5: note = peptide
SGNRQ

SEQ ID NO: 282 - protein, 5 aa, synthetic construct
  REGION 1..5: note = peptide
SGNRF

SEQ ID NO: 283 - protein, 5 aa, synthetic construct
  REGION 1..5: note = peptide
SGNRH

SEQ ID NO: 284 - protein, 5 aa, synthetic construct
  REGION 1..5: note = peptide
SGNRR

SEQ ID NO: 285 - protein, 5 aa, synthetic construct
  REGION 1..5: note = peptide
SGNRK

SEQ ID NO: 286 - protein, 5 aa, synthetic construct
  REGION 1..5: note = peptide
SGNRC

SEQ ID NO: 287 - protein, 5 aa, synthetic construct
  REGION 1..5: note = peptide
SGNRL

SEQ ID NO: 288 - protein, 5 aa, synthetic construct
  REGION 1..5: note = peptide
SGNRY

SEQ ID NO: 289 - protein, 5 aa, synthetic construct
  REGION 1..5: note = peptide
SGNRN

SEQ ID NO: 290 - protein, 5 aa, synthetic construct
  REGION 1..5: note = peptide
SGNRI

SEQ ID NO: 291 - protein, 5 aa, synthetic construct
  REGION 1..5: note = peptide
SGNRD

SEQ ID NO: 292 - protein, 5 aa, synthetic construct
  REGION 1..5: note = peptide
SGNRW

SEQ ID NO: 293 - protein, 5 aa, synthetic construct
  REGION 1..5: note = peptide
SGNRE

SEQ ID NO: 294 - protein, 5 aa, synthetic construct
  REGION 1..5: note = peptide
SGNRV

SEQ ID NO: 295 - protein, 5 aa, synthetic construct
  REGION 1..5: note = peptide
SGNRM

SEQ ID NO: 296 - protein, 5 aa, synthetic construct
  REGION 1..5: note = peptide
SGNRA

SEQ ID NO: 297 - protein, 5 aa, synthetic construct
  REGION 1..5: note = peptide
SGNRT

SEQ ID NO: 298 - protein, 5 aa, synthetic construct
  REGION 1..5: note = peptide
SGNRP

SEQ ID NO: 299 - protein, 5 aa, synthetic construct
  REGION 1..5: note = peptide
SMHRQ

SEQ ID NO: 300 - protein, 5 aa, synthetic construct
  REGION 1..5: note = peptide
SMHRF

SEQ ID NO: 301 - protein, 5 aa, synthetic construct
  REGION 1..5: note = peptide
SMHRH

SEQ ID NO: 302 - protein, 5 aa, synthetic construct
  REGION 1..5: note = peptide
SMHRR

SEQ ID NO: 303 - protein, 5 aa, synthetic construct
  REGION 1..5: note = peptide
SMHRK

SEQ ID NO: 304 - protein, 5 aa, synthetic construct
  REGION 1..5: note = peptide
SMHRC

SEQ ID NO: 305 - protein, 5 aa, synthetic construct
  REGION 1..5: note = peptide
SMHRL

SEQ ID NO: 306 - protein, 5 aa, synthetic construct
  REGION 1..5: note = peptide
SMHRY

SEQ ID NO: 307 - protein, 5 aa, synthetic construct
  REGION 1..5: note = peptide
SMHRN

SEQ ID NO: 308 - protein, 5 aa, synthetic construct
  REGION 1..5: note = peptide
SMHRI

SEQ ID NO: 309 - protein, 5 aa, synthetic construct
  REGION 1..5: note = peptide
SMHRD

SEQ ID NO: 310 - protein, 5 aa, synthetic construct
  REGION 1..5: note = peptide
SMHRW

SEQ ID NO: 311 - protein, 5 aa, synthetic construct
  REGION 1..5: note = peptide
SMHRE

SEQ ID NO: 312 - protein, 5 aa, synthetic construct
  REGION 1..5: note = peptide
SMHRV

SEQ ID NO: 313 - protein, 5 aa, synthetic construct
  REGION 1..5: note = peptide
SMHRM

SEQ ID NO: 314 - protein, 5 aa, synthetic construct
  REGION 1..5: note = peptide
SMHRA

SEQ ID NO: 315 - protein, 5 aa, synthetic construct
  REGION 1..5: note = peptide
SMHRT

SEQ ID NO: 316 - protein, 5 aa, synthetic construct
  REGION 1..5: note = peptide
SMHRP

SEQ ID NO: 317 - protein, 5 aa, synthetic construct
  REGION 1..5: note = peptide
SLLRQ

SEQ ID NO: 318 - protein, 5 aa, synthetic construct
  REGION 1..5: note = peptide
SLLRF

SEQ ID NO: 319 - protein, 5 aa, synthetic construct
  REGION 1..5: note = peptide
SLLRH

SEQ ID NO: 320 - protein, 5 aa, synthetic construct
  REGION 1..5: note = peptide
SLLRR

SEQ ID NO: 321 - protein, 5 aa, synthetic construct
  REGION 1..5: note = peptide
SLLRK

SEQ ID NO: 322 - protein, 5 aa, synthetic construct
  REGION 1..5: note = peptide
SLLRC

SEQ ID NO: 323 - protein, 5 aa, synthetic construct
  REGION 1..5: note = peptide
SLLRL

SEQ ID NO: 324 - protein, 5 aa, synthetic construct
  REGION 1..5: note = peptide
SLLRY

SEQ ID NO: 325 - protein, 5 aa, synthetic construct
  REGION 1..5: note = peptide
SLLRN

SEQ ID NO: 326 - protein, 5 aa, synthetic construct
  REGION 1..5: note = peptide
SLLRI

SEQ ID NO: 327 - protein, 5 aa, synthetic construct
  REGION 1..5: note = peptide
SLLRD

SEQ ID NO: 328 - protein, 5 aa, synthetic construct
  REGION 1..5: note = peptide
SLLRW

SEQ ID NO: 329 - protein, 5 aa, synthetic construct
  REGION 1..5: note = peptide
SLLRE

SEQ ID NO: 330 - protein, 5 aa, synthetic construct
  REGION 1..5: note = peptide
SLLRV

SEQ ID NO: 331 - protein, 5 aa, synthetic construct
  REGION 1..5: note = peptide
SLLRM

SEQ ID NO: 332 - protein, 5 aa, synthetic construct
  REGION 1..5: note = peptide
SLLRA

SEQ ID NO: 333 - protein, 5 aa, synthetic construct
  REGION 1..5: note = peptide
SLLRT

SEQ ID NO: 334 - protein, 5 aa, synthetic construct
  REGION 1..5: note = peptide
SLLRP

SEQ ID NO: 335 - protein, 8 aa, synthetic construct
  REGION 1..8: note = Peptide
AIPMSIPP

SEQ ID NO: 336 - protein, 8 aa, synthetic construct
  REGION 1..8: note = Peptide
AIPRSIPP

SEQ ID NO: 337 - protein, 8 aa, synthetic construct
  REGION 1..8: note = Peptide
AIARSIPP

SEQ ID NO: 338 - protein, 8 aa, synthetic construct
  REGION 1..8: note = Peptide
SMTRSIPP

SEQ ID NO: 339 - protein, 8 aa, synthetic construct
  REGION 1..8: note = Peptide
SMTRVVGG

SEQ ID NO: 340 - protein, 8 aa, synthetic construct
  REGION 1..8: note = Peptide
SMTRGVGG

SEQ ID NO: 341 - protein, 8 aa, synthetic construct
  REGION 1..8: note = Peptide
SGQRSIPP

SEQ ID NO: 342 - protein, 8 aa, synthetic construct
  REGION 1..8: note = Peptide
SVTRSIPP

SEQ ID NO: 343 - protein, 8 aa, synthetic construct
  REGION 1..8: note = Peptide
SATRSIPP

SEQ ID NO: 344 - protein, 8 aa, synthetic construct
  REGION 1..8: note = Peptide
SFNRSIPP

SEQ ID NO: 345 - protein, 8 aa, synthetic construct
  REGION 1..8: note = Peptide
SWKKSIPP

SEQ ID NO: 346 - protein, 8 aa, synthetic construct
  REGION 1..8: note = Peptide
SEARSIPP

SEQ ID NO: 347 - protein, 8 aa, synthetic construct
  REGION 1..8: note = Peptide
SLLRSIPP

SEQ ID NO: 348 - protein, 8 aa, synthetic construct
  REGION 1..8: note = Peptide
SLLRVIPP

SEQ ID NO: 349 - protein, 8 aa, synthetic construct
  REGION 1..8: note = Peptide
SLLRIIPP

SEQ ID NO: 350 - protein, 8 aa, synthetic construct
  REGION 1..8: note = Peptide
SVVKSIPP

SEQ ID NO: 351 - protein, 8 aa, synthetic construct
  REGION 1..8: note = Peptide
SDYKSIPP

SEQ ID NO: 352 - protein, 8 aa, synthetic construct
  REGION 1..8: note = Peptide
SVRKSIPP

SEQ ID NO: 353 - protein, 8 aa, synthetic construct
  REGION 1..8: note = Peptide
SPRRSIPP

SEQ ID NO: 354 - protein, 8 aa, synthetic construct
  REGION 1..8: note = Peptide
SMDRSIPP

SEQ ID NO: 355 - protein, 8 aa, synthetic construct
  REGION 1..8: note = Peptide
SLGRSIPP

SEQ ID NO: 356 - protein, 8 aa, synthetic construct
  REGION 1..8: note = Peptide
SKGRSIPP

SEQ ID NO: 357 - protein, 8 aa, synthetic construct
  REGION 1..8: note = Peptide
SGWRSIPP

SEQ ID NO: 358 - protein, 8 aa, synthetic construct
  REGION 1..8: note = Peptide
SYARSIPP

SEQ ID NO: 359 - protein, 8 aa, synthetic construct
  REGION 1..8: note = Peptide
SMHRSIPP

SEQ ID NO: 360 - protein, 8 aa, synthetic construct
  REGION 1..8: note = Peptide
SNSRSIPP